SEQUENCE LISTING (8 sequences)

SEQ ID NO: 1 - protein, 29 aa, Homo sapiens
ALDAAYCFRNVQDNCCLRPLYIDFKRDLG

SEQ ID NO: 2 - protein, 16 aa, Homo sapiens
CLRPLYIDFKRDLGWK

SEQ ID NO: 3 - protein, 16 aa, Artificial Sequence
  Analogue of TGF-beta fragment, consensus
XXRXLYIDFXXDLGWK

SEQ ID NO: 4 - protein, 16 aa, Artificial Sequence
  TGF-beta fragment
CVRQLYIDFRKDLGWK

SEQ ID NO: 5 - protein, 20 aa, Artificial Sequence
  Analogue of TGF-beta fragment
RNLEENCCVRPLYIDFRQDL

SEQ ID NO: 6 - protein, 16 aa, Artificial Sequence
  Analogue of TGF-beta fragment
CXRXLYIDFXXDLGWK

SEQ ID NO: 7 - protein, 16 aa, Artificial Sequence
  Analogue of TGF-beta fragment
CVRXLYIDFRXDLGWK

SEQ ID NO: 8 - protein, 18 aa, Artificial Sequence
  Canine parvovirus epitope
CDGAVQPDGGQPAVRNER

(19) United States
(12) Patent Application Publication
(10) Pub. No.: US 2002/0160012 A1
(43) Pub. Date: Oct. 31, 2002

(54) Vaccine chip technology exploiting immunostimulating fragment of TGF-BETA

(76) Inventor: Peter Kaastrup, Maaloev (DK)

Correspondence Address: BROWDY AND NEIMARK, P.L.L.C., 624 NINTH STREET, NW, SUITE 300, WASHINGTON, DC, 20001-5303, US

(21) Appl. No.: 09/822,873

(22) Filed: Apr. 2, 2001

Related U.S. Application Data
(60) Provisional application No. 60/246,973, filed on Nov. 13, 2000.

(30) Foreign Application Priority Data
Mar. 31, 2000 (DK) PA 2000 00540

(51) Int. Cl. A61K 39/00

(52) U.S. Cl. 424/185.1

(57) ABSTRACT

There is provided an immunogenic composition comprising at least one fragment of TGF-^{β} capable of eliciting an immunostimulating effect in an individual, and at least one immunogenic determinant against which it is desirable to elicit an immunogenic response, wherein said at least one fragment of TGF-^{β} and said at least one immunogenic determinant are not identical. Following immunisation, the effect exerted by the TGF-^{β} fragment according to the invention results in the generation of an increased immunological response against the immunogenic determinant. By loading otherwise low immunogenic determinants into the vaccine chip according to the invention, thus generating an immunogenic composition according to the present invention, the immunogenic determinants will acquire an increased immunogenicity. There is also provided a vaccine formulation and a method for immunising an individual with the immunological compositions according to the invention.

### FIELD OF THE INVENTION

The present invention relates to immunogenic compositions comprising at least one fragment of TGF-β capable of eliciting an immunostimulating effect in an individual, and at least one immunogenic determinant against which it is desirable to elicit an immunogenic response

### BACKGROUND OF THE INVENTION

Transforming growth factor-beta (TGF-β) is a 25 kd dimer polypeptide that acts as a multifunctional cytokine in the regulation of a wide variety of cellular functions including cell growth and differentiation. TGF-β is secreted by virtually all cell types in an inactive form. This latent form can be activated by proteolytic cleavage of mature TGF-β from its precursor (at the Arg-Ala bond in position 278).

Three forms of TGF-β; TGF-β1, TGF-β2, and TGF-β3 have been identified in mammals, and methods for purifying TGF-β from various species such as human, mouse, green monkey, pig, bovine, and chick, and from various body sources such as bone, platelets, or placenta, are known. Methods for producing TGF-β in recombinant cell culture and for determining its activity have also been described (U.S. Pat. No. 5,061,786, and references incorporated therein).

The involvement of TGF-β in both stimulation and inhibition of cellular proliferation, cellular differentiation, and other essential cell function processes, has previously been described (M. Spom, Science, 233:532,1986). TGF-β has pro-inflammatory activities as well as immuno-suppressive activities, and studies have indicated a role for TGF-β in cells of the immune system It is known that TGF-β is capable of suppressing the production of cytokine (e.g., IFN-γ, TNF-α), and TGF-β has also been used as an immuno-suppressant for treating inflammatory disorders and as a promoter of cachexia. TGF-β also induces collagen secretion in human fibroblast cultures, stimulates the release of prostaglandins and the mobilization of calcium, and inhibits endothelial regeneration. (U.S. Pat. No. 5,061,786 and references incorporated therein).

TGF-β is capable of opposing the action of various other growth factors, and TGF-β has been reported to inhibit the growth of many cell types including cells of the immune system (see fx Roberts, A. B., Molecular and cell biology of TGF-beta, Miner. Electrolyte Metab., 24(2-3), pp. 111-119, [1998]). TGF-^{β} has also been found to suppress the expression of Class II histocompatibility antigens on human cells induced by human interferon-γ.

TGF-β exerts its diverse biological effects through receptors on a cell surface capable of binding to TGF-β. TGF-β receptors are widely distributed and expressed by most cell types. Currently three types of receptors have been identified based on their high-affinity binding to radiolabelled TGF-β. Type I and II are transmembrane spanning proteins, and type III is known as a betaglycan receptor type. The action and interaction of the different receptor types are complex and not fully understood. Studies have shown that receptor type II only binds TGF-β in the presence of receptor type I. Type I and II are referred to as signalling receptors since they mediate the incoming signal associated with ligand binding through an intracellular signal transduction pathway. The function of type Ill receptors are to present TGF-β to the signalling receptors (William, P. E., Fundamental Immunology, 1999, 4^{th} ed., Lippincott-Raven Publishers).

The human immune system comprises numerous different types of cells that have complex, multiple functions-and form interconnected relationships. The humoral antibody is a major component of the immune system, and it plays an essential role in protecting a host organism against an infection by foreign organisms such as vira or microbial cells.

Antibodies, also known as immunoglobulins, are protein molecules which have exquisite specificity for a foreign organism that stimulates their production. Immunoglobulins (Ig) are a class of structurally related proteins consisting of pairs of polypeptide chains linked together by disulfide bonds. There are five Ig isotypes: IgA, IgM, IgD, IgE, and IgG (with four subclasses IgG1, IgG2, IgG3, and IgG4 for humans and mice).

Not all antibody isotypes are equally well suited for performing the many diverse activities associated with the functionality of Ian antibody. IgA is primarily present in secreted body fluids such as tears, urine, saliva, colostrum, sweat, and mucus (i.e., secretory IgA). IgA is believed to be the primary immunological defense against local infections in such areas as the respiratory or gastrointestinal tract. Secretory IgA is also an efficient agglutinating antibody as well as an efficient antiviral antibody capable of preventing a virus from entering a host cell.

The actions of the very versatile and long-lived IgG ranges from toxin neutralization to activation of complement and opsonization. For example, IgG reacts with epitopes on an invading microorganism via its Fab portions, and this interaction leads to the final engulfing and destruction of the microorganism in question. IgG also plays an important role in antibody-dependent, cell-mediated cytotoxicity (ADCC) by activating natural killer cells capable of destroying a target object by releasing various cell killing substances. IgG is also an effective virus neutralizing antibody.

IgM antibodies are not as versatile as IgG; they are poor toxin-neutralizing antibodies, and they are not as efficient in the neutralization of vira. IgM is found predominantly in the intravascular spaces and is the isotype synthesized by children and adults in appreciable amounts after immunization or exposure to T-independent antigens. IgM is the first isotype that is synthesized after immunization with T-dependent antigens. IgM molecules are the most efficient agglutinating and complement-activating antibodies.

IgD molecules are present on the surface of B lymphocytes and appear to be involved in the differentiation of these cells. IgD is described as not being associated with a generally protective function.

IgE, also called reaginic antibody, is of paramount importance in hypersensitivity or allergy reactions. These reactions may be mild, such as in the case of a mosquito bite, or severe, as in the case of bronchial asthma. The reactions may even result in systemic anaphylaxis, which can cause death within minutes.

Despite the above-illustrated variability, all immunoglobulins are derived from antibody-secreting cells. The precursors of antibody-secreting cells are B lymphocytes, also known as “B cells”. B cells are a type of lymphocyte and are derived from hematopoietic stem cells by a complex set of differentiation events that are only partially understood.

B cells bear as a cell-surface receptor an immunoglobulin (Ig) molecule specialized for expression on the cell surface. Newly differentiated B cells initially express surface Ig solely of the IgM class. Maturation of a B cell leads to the appearance of other immunoglobulin isotypes on the surface of the B cell.

B cells must be activated in order to release antibodies. There are many ways to activate B cells, including cross-linkage of membrane Ig molecules by the antigen (cross-linkage-dependent B cell activation), direct encounter with T cells (helper T cells or helper T cell-associated molecules, such as CD40 ligand), or encounter with mitogens. During or following such encounters, the antigen presents epitopes recognized by Ig molecules on the surface of B cells.

Once B cells expressing IgM and/or IgD on their surface are suitably activated the antibodies that are initially produced are primarily of the IgM isotype. In response to a specific challenge, T helper cells allow B cells to “class switch”. A B cell that initially expresses receptors of the IgM and IgD classes may differentiate into a cell that expresses IgG, IgA, and/or IgE receptors, and the B cell may subsequently differentiate further into a cell that secretes those antibodies. This process results in the production of different antibodies with distinct biological functions against the antigen responsible for inducing the antibody response.

The induction of the switching process depends upon the action of a specialized set of B cell stimulants. Studies have identified two different kinds of stimuli that are important for the induction of isotype switching. The first kind of stimuli are predominantly responsible for the specificity of the isotype-switch event. These include, but are not limited to, the cytokine IL-4, interferon-γ (IFN-γ) and TGF-β. Prior art examples of how TGF-β is involved in isotype Ig switching to IgA is disclosed in U.S. Pat. No. 5,874,085.

Vaccines are preparations of immunogenic material for administration to induce in the recipient an immunity to infection or intoxication by a given infecting agent. Vaccines may be prepared from viruses, rickettsiae, bacteria, protozoa and metazoa. Vaccines may be sterile suspensions of the killed organisms, of toxoids or other synthetic immunogenic material derived from the organisms or recombinant sources, which can be administered by intravenous injection or through oral, nasal and/or mucosal administration. Vaccines may be either simple vaccines prepared from one species of organism or a variety of organisms, or they may be mixed vaccines containing two or more simple vaccines. They are prepared in such a manner as not to destroy the immunogenic material, although the methods of preparation vary, depending on the vaccine.

The chain of events leading to an immune response is initiated by an uptake of an antigen by an antigen-presenting cell (APC), which degrades and re-express processed parts of the antigen in the context of self MHC molecules. T-cells then recognize the newly formed MHC-peptide complex, and cellular activation is triggered by the binding of antigen to the T cell receptor (TCR), forming an antigen/TCR complex which transduces the antigen-specific extracellular stimulation across the plasma membrane and generates intracellular signals. The processed peptide fragments are referred to as T-cell epitopes. However, some epitopes lack the structural elements to bind MHC molecules and stimulate specific T cells. This problem can be circumvented by chemically linking of the epitope with a carrier molecule. Many studies have shown that carrier molecules are important in the immunisation process, such as in vaccines. They are used to enhance the immunogenic response of antigens. Many carrier molecules will induce an antibody response to itself when used for an initial immunisation. This may interfere with the induction of an effective antibody response to a given immunogen when it is inoculated conjugated to the carrier.

The carrier molecules can be conjugated to the antigen e.g. by covalent linkage. However, studies by Sarobe et al. (Sarobe, P., Lasarte, José, J., Golvano, J., Gullón, A., Civeira, M. P., Priesto, J. and Borrás-Cuesta, F., Eur. J. Immunol., 1991, 21; pp. 1555-1558) suggest that the induction of antibodies against a peptide hapten does not require covalent linkage between the hapten and a immonogenic peptide. The results suggest that the need of coupling a peptide to a carrier molecule to obtain anti-peptide antibodies could be bypassed.

Further, Partidos et al. (Partidos, C. D., Obeid, O. E. and Steward, M. W., Immunology, 1992, 77, pp. 262-266) describe achieving an antibody response to synthetic peptides when employing co-immunisation of non-conjugated peptides representing B- and T-cell epitopes. The results indicate that simple co-immunization of peptides representing B-cell epitopes with peptides representing Th determinants results in the production of antibody to the B-cell epitopes without the requirement for the covalent linkage of the B- and Th-cell epitopes.

The conjugation of protein carriers to TGF-β antagonists is described by Huang et al. (Huang S. S., Iiu, Q., Johnson, F. E., Konish, Y. and San, J., 1997, JBC online, 272, 43, pp. 27155-27159) wherein TGF-β antagonists are converted into partial agonists by conjugation to protein carriers in mink lung epithelial cells.

Adjuvants are often used to optimise the efficacy of an immunogenic composition. Adjuvants generally consist of agents that are included in the formulation used to provide and/or enhance the ability of the immunogenic composition to induce a desired immune response.

The contribution of TGF-β in immunological processes has so far been described as being primarily of an immuno-suppressant character or as being involved in Ig-class switching as mentioned above,

Due to its ability to act as a suppressor of cytokine production TGF-β has been used as an immunosuppressant for treating inflammatory disorders. TGF-β has been found to suppress the expression of Class II histocompatibility antigens on human cells induced by human interferon-γ. Furthermore, TGF-β is described as suppressing the immune system, and U.S. Pat. No. 5,772,995 relates to a method for enhancing tumor cell immunity by preventing or reducing the expression of immunosuppressive agents, such as TGF-β.

The association of TGF-β with immunological activities is also described by Wrane (Wrana, J. L.; TGF-β receptors and signalling mechanisms; Miner Electrolyte Metab, 24(2-3), pp 120-130, 1998) who discloses how the knock-out of TGF-β genes cause a breakdown of the immune homeostasis. TGF-β is known to inhibit the activation of T-cells and monocytes, as well as promoting class switching to IgA. Additionally TGF-β is known to inhibit the proliferation of B lymphocytes, to suppress the expression by activated B-lymphocytes of membrane immunoglobulin, and to decrease secretion of IgG and IgM. Accordingly, TGF-β is often referred to as an immunosuppressant in the art.

However, recently three isoforms of TGF-β have been described as having stimulatory effects on human T-cells. Schlött et al. (Schlött, A., Sj\overline{o}gren, H. O. and Lindvall, M., Scand. J. Immunol., 1998, 48, pp. 371-378) disclose how in vitro experiments with cell cultures showed how the three different isozymes increased the proliferative response of rat T lymphocytes upon exposure of the antigen staphylococcal enterotoxin A, which was also accompanied by a lower percentage of apoptotic cells.

Fragments of TGF-β have previously been described. EP 290 012 discloses fragments of TGF-β2 having at least about eight amino acids, for example in the region of N-terminal amino acids 1 to 20, particularly 4-15, and more particularly 9-14, a C-terminal sequence, or a truncated N-terminal or C-terminal molecule. Further fragments of TGF-β are disclosed in EP 267 463, WO 91/05565, and WO 94/17099.

Van den Eijden-Van Raaij et al. (J. Immunological Meth., 1990, vol. 133, p. 107-118) have disclosed a 29 amino acid fragment of TGF-β. Further, Jin et al. (J. Prot. Chem., 1991, Vol,. 10(5), p. 565-575) disclose the separation, purification and sequence identification of TGF-β from bovine milk. In WO 88/05788 a growth factor having TGF-β activity having various N-terminal sequences are disclosed. U.S. Pat. Nos. 5,061,786, 5,268,455, and 5,118,791 disclose biologically active polypeptides based on TGF-β sequences. The disclosed sequences are all related to the use of TGF-β as an immunosuppressant, and as an immunomodulator having transforming growth factor-beta-like activity. It is described how the sequences may be applied in a method for the production of antibodies neutralizing immunosuppressive proteins, i.e. TGF-β, or for ameliorating an immune or inflammatory disorder.

The above references do not reveal a TGF-β fragment in combination with an immunogenic determinant against which it is desirable to raise an immunogenic response.

### SUMMARY OF THE INVENTION

It is one purpose of the present invention to provide a vaccine chip technology that exploits an immunostimulating fragment of TGF-β. Thus, the present invention relates to an immunogenic composition comprising

i) at least one fragment of TGF-β capable of eliciting an immunostimulating effect in an individual, and

ii) at least one immunogenic determinant against which it is desirable to elicit an immunogenic response,

wherein said at least one fragment of TGF-P and said at least one immunogenic determinant are not identical.

Further the invention concerns a vaccine comprising the immunogenic composition described by the invention.

Another object of the present invention is to provide a fragment of TGF-β capable of facilitating an immunostimulating effect in an individual for use as a medicament.

Yet another aspect of the present invention relates to a method of immunising an individual in need of immunisation, said method comprising the steps of:

i) providing an immunogenic composition or a vaccine according to the present invention, and

ii) administering said immunogenic composition or said vaccine to said individual.

The invention also discloses the use of a fragment of TGF-β capable of facilitating an immunostimulating effect in an individual in the manufacture of an immunogenic composition or a vaccine.

### FIGURES

FIG. 1 illustrates the enhancing effect of TGF-29 on the immune response of 3 rabbits (#1-3) after experimental immunisation with the low immunogenic peptide “parv”. The immune response is indicated as the difference in ELISA reader measured milli absorbance between an immune serum after immunisation and the corresponding T₀ serum before immunisation, at each serial twofold sera dilution.

Figure illustrates the immune response of 2 rabbits (#264 and #265) over time after experimental, immunisation with a TGF29-Parvo virus peptide conjugate. A) ELISA plates wherein TGF29 or “parv” were used as targets. Dark colour indicates a high antibody titer against the target in the serum sample. B) The immune response is indicated as the difference in ELISA reader measured milli absorbance between an immune serum (T₁, T₂, T₃ or T₄ ) after immunisation and the corresponding T₀ serum before immunisation, at three different 5-fold sera dilution (starting at 20% anti sera(as)). The immunesera T₀-T₅ were obtained as bi-weekly bleedings.

FIG. 3 illustrates the dose response effect of TGF-29 on the immune response of rabbits (#242, #243, #244 and #245) to the immusied with a constant dosis of the parvo virus peptide “parv” together with Titermax adjuvant. The immune response is shown as difference in ELISA reader measured milli absorbance between an immune serum (T₃) after immunisation and the corresponding T₀ serum before immunisation, at 3 serial five-fold antisera dilution (starting at 20% antisera (as) dilution) The T3 immunewera were collected 6 weeks after the first immunisation.

FIG. 4 illustrates the effect of TGF-29 on survival of Atlantic salmon on day 17 after challenge with infectious pathogen (Aeromonas salmonicida), when the salmon had been vaccinated with either diluent alone, TGF29 alone, rAsOMP or rAsOMP together with TGF29 prior to the challenge.

### DEFINITIONS

Adjuvant:

Any substance whose admixture with an administered immunogenic determinant increases or otherwise modifies the immune response to said determinant.

Amino Acid:

Any synthetic or naturally occurring amino carboxylic acid, including any amino acid occurring in peptides and polypeptides including proteins and enzymes synthesized in vivo.

Antibody:

Immunoglobulin molecule or immunologically active portion thereof, i.e. molecules that contain an “antigen binding site” or paratope. An antigen binding site is that structural portion of an antibody molecule that specifically binds to an antigen at a B cell epitope.

Antibody Response:

Response at least involving the binding of molecularly distinct Ig molecules to different epitopes present on at least one antigen.

Antigenic:

Functionality associated with a molecule capable of eliciting an antibody response.

Antigenic Determinant:

A molecule, or a part thereof, containing one or more epitopes that will elicit an antibody response in a host organism.

Carrier Protein:

A scaffold structure, e.g. a polypeptide or a polysaccharide, to which an immunogenic determinant is capable of being associated.

Complement:

A complex series of blood proteins whose action “complements” the work of antibodies. Complement destroys bacteria, produces inflammation, and regulates immune reactions.

Conjugated:

An association formed between an immunogenic determinant and a carrier. The association may be a physical association generated e.g. by the formation of a chemical bond, such as e.g. a covalent bond, formed between the immunogenic determinant and the carrier.

Co-immunisation:

Immunisation by means of separate and/or sequential administration to an individual of an immunogenic determinant and a carrier.

Conservative Amino Acid Substitution:

Substitution of one amino acid (within a predetermined group of amino acids) for another amino acid (within the same group) exhibiting similar or substantially similar characteristics. Within the meaning of the term “conservative amino acid substitution” as applied herein, one amino acid may be substituted for another within groups of amino acids characterised by having

i) polar side chains (Asp, Glu, Lys, Arg, His, Asn, Gln, Ser, Thr, Tyr, and Cys,)

ii) non-polar side chains (Gly, Ala, Val, Leu, Ile, Phe, Trp, Pro, and Met)

iii) aliphatic side chains (Gly, Ala Val, Leu, Ile)

iv) cyclic side chains (Phe, Tyr, Trp, His, Pro)

v) aromatic side chains (Phe, Tyr, Trp)

vi) acidic side chains (Asp, Glu)

vii) basic side chains (Lys, Arg, His)

viii) amide side chains (Asn, Gin)

ix) hydroxy side chains (Ser, Thr)

x) sulphor-containing side chains (Cys, Met), and

xi) amino acids being monoamino-dicarboxylic acids or monoamino-monocarboxylic-monoamidocarboxylic acids (Asp, Glu, Asn, Gln).

Cytokine:

Growth or differentiation modulator, used non-determinative herein, and should not limit the interpretation of the present invention and claims. In addition to the cytokines, adhesion or accessory molecules, or any combination thereof, may be employed alone or in combination with the cytokines.

Cytotoxic response: T-cell mediated destruction of a target cell.

Effective Amount:

An effective amount of an immunostimulating fragment of TGF-β sufficient to enhance a humoral and/or cellular immune response induced by an immunogenic composition including a vaccine.

Epitope:

A specific site on a protein to which only certain antibodies bind.

Functional equivalents: Functional equivalents of a fragment of TGF-β comprising a predetermined amino acid sequence are defined as:

i) fragments comprising an amino acid sequence capable of being recognised by an antibody also capable of recognising the predetermined amino acid sequence, and/or

ii) fragments comprising an amino acid sequence capable of binding to a receptor moiety also capable of binding the predetermined amino acid sequence, and/or

iii) fragments having at least a substantially similar immunostimulating effect as the fragment of TGF-β comprising said predetermined amino acid sequence.

Hapten:

A compound, usually of low molecular weight, that is not in itself immunogenic but that, after administration with a carrier protein or cells (either conjugated or non-conjugated), becomes immunogenic and induces an antibody response resulting in antibody binding of the hapten in the absence of carrier.

Immunization:

Process of inducing an immunological response in an organism.

Immunogenic determinant:

A molecule, or a part thereof, containing one or more epitopes that will stimulate the immune system of a host organism to make a secretory, humoral and/or cellular antigen-specific response, or to a DNA molecule which is capable of producing such an immunogen in a vertebrate.

Immunological Response:

Response to a immunogenic composition comprising an immunogenic determinant. An immune response involves the development in the host of a cellular- and/or antibody-mediated response to the administered composition or vaccine in question. An immune response generally involves the action of one or more of i) the antibodies raised, ii) B cells, iii) helper T cells, iv) suppressor T cells, and v) cytotoxic T cells, directed specifically to an immunogenic determinant present in an administered immunogenic composition.

Immunogenic Composition:

Composition capable of raising an immunological response in an individual.

Immunogenic:

Functionality associated with an entity capable of eliciting an immunological response.

Immunostimulating Effect:

Functionality associated with an entity capable of eliciting an enhanced immune response. An enhanced immune response will be understood within the meaning of the observed difference in the immune response measured as an enhancement of an antibody production and/or a cytotoxic T-cell activity, or otherwise registered, when an immunogenic composition is administered in the presence or absence, respectively, of the entity. An immunogenic composition comprising the entity will be understood as being a composition according to the present invention.

Individual:

Any species or subspecies of bird, mammal, fish, amphibian, or reptile.

Peptide:

Refers to molecule comprising at least two amino acids.

Systemic Immune Response:

An immune response which is not localized, but affects the immunised individual as a whole, thus allowing specific subsequent responses to the same stimulus.

TGF-β:

Transforming Growth Factor Beta (β). It will be understood that the term TGF-β as used herein includes any variant and/or functional equivalent thereof, unless the contrary is indicated

Treatment:

Administration to an individual of an immunogenic composition according to the invention and yielding, when in the form of a vaccine, a protective immune response. Treatment includes prophylaxis and/or therapy.

Vaccination:

Process of inducing a protective immune response in an organism.

Vaccine:

Immunogenic composition capable of raising a protective immune response in a subject,

Vaccine Chip Technology:

Technology that exploits an immunostimulating fragment of TGF-β.

Variant:

A functional equivalent of TGF.

### DETAILED DESCRIPTION OF THE INVENTION

It is one aim of the present invention to provide an immunogenic composition comprising an immunogenic determinant against which an increased immunogenicity is elicited during or following an immunisation of an individual.

The below presently preferred hypotheses illustrate aspects of the present invention and serve to provide a conceivable explaination as to the effects obtained with the immunogenic compositions according to the invention.

According to one hypothesis, it is believed that a natural immunosuppressing cytokine and/or lymphokine activity may be downregulated locally as a result of using the immunogenic composition according to the present invention.

Without being bound by theory it is believed that the present invention exerts on the immune system of an individual the positive effect associated With a TGF-β fragment capable of eliciting an immunostimulating effect in an individual.

Following immunisation, the effect exerted by the TGF-β fragment according to the invention results in the generation of an increased level of antibodies against the immunogenic determinant. The increased level of antibodies is believed to be the result of the binding or association of at least one immunosuppressing cytokine of the individual to the antibodies raised.

Consequently, by loading otherwise low immunogenic determinants into the vaccine chip according to the invention, thus generating an immunogenic composition according to the present invention, the immunogenic determinants will acquire an increased immunogenicity.

It is thus believed that when an individual is immunised with a composition comprising the TGF-β fragment of the invention, antibodies against the fragment are generated. After immunisation the antibodies may—in addition to binding to the TGF-β fragment—also bind to naturally occurring, immunosuppressing cytokines of the immunised individual. Assuming that naturally occurring cytokines under normal circumstances exerts an inhibiting effect on the immune response, the binding of antibodies to the naturally occurring cytokines is believed to result in blocking and/or reducing the inhibitory effect of naturally occurring cytokines on the immune system.

The blocking of the inhibiting effect of naturally occurring cytokines on the immune system may be especially effective locally in lymph nodes near the immunisation site. When specialised B-cells with surface localised antibodies against the fragment of TGF-β are recruited or induced, they are especially abundant at the immunisation site. Both the antibodies secreted in the body fluid and the antibodies on the B-cells (surface immunoglobulins) are believed to contribute to an effective binding to the naturally occurring cytokines. This reduces or prevents inhibition of local cells by natural cytokines.

In a second embodiment of the present invention the fragment of TGF-β capable of eliciting an immunostimulating effect binds to or associates with the cytokine receptors of the immunised individual.

When an individual is immunised with an immunogenic composition according to the present invention, the fragment of TGF-β may contact the same receptors as naturally occurring cytokines. According to the invention it is envisioned that the fragment of TGF-β occupies receptor sites of natural cytokines including TGF.

The fragment of TGF-β may interact with the receptors alone, or it may interact with substances or co-factors important for inhibiting the function of natural cytokines present in the immune system. The effect of the fragment of TGF-β on the immune response may be caused by an altered functionality of the membrane receptors important for cellular uptake of natural cytokines. It may also be due to the indirect effect on the catabolism (degradation) of either the receptors or the natural cytokines.

Accordingly, the molecular mechanism behind the effect on the immune response that is directly or indirectly caused by the fragment of TGF-β comprised in the immunogenic compositions according to the present invention may be due to a functional downregulation of the inhibiting effect of natural cytokines. Furthermore, the possibility that an enhanced effect on the immune response against immunogenic determinants present in the composition is not ruled out.

In a third hypothesis the fragment of TGF-^{β} capable of eliciting an immunostimulating effect is capable of associationg with or fits into the MHC molecules of an immunised individual. It is believed that a high immune response against a target immunogen normally requires that one or more fragments of the target immunogen (or epitope thereon are presented to T-helper cells by MHC-molecules present on the surface of antigen-presenting cells. The immunogenic determinants are thereby recognised by the T-helper cell receptors.

In the event that no fragments fit into the MHC molecules there will be no help from the T-cells, and the resulting immune response may be low. Smal, low immunogenic molecules, such as most oligo-peptides is believed not to possess the required structure to fit into the MHC molecules. It is therefore necessary to attach such smaller peptides to a carrier species.

In the organism of the individual the carrier-peptide complex is believed to be degraded into fragments, some of which are likely to fit into the MHC molecules. In this way the necessary help from T-cells is mobilised in the local environment This may lead to a state that will cause a high immune response to otherwise low immunogenic molecules upon a first exposure and optionally also upon a second exposure.

### PREFERRED EMBODIMENTS OF THE INVENTION

The present invention in one embodiment pertains to an immunogenic composition comprising a fragment of TGF-^{β} comprising the amino acid sequence

| | |
|---|---|
| Ala-Leu-Asp-Ala-Ala-Tyr-Cys-Phe-Arg-Asn-Val-Gln-Asp-Asn | (SEQ ID NO: 1) |
| -Cys-Cys-Leu-Arg-Pro-Leu-Tyr-Ile-Asp-Phe-Lys-Arg-Asp- | |
| Leu-Gly | |

including any functional equivalents thereof obtained by addition, substitution or deletion of at least one amino acid

In another embodiment the fragment of TGF-^{β} essentially consists of the amino acid sequence

| | |
|---|---|
| Ala-Leu-Asp-Ala-Ala-Tyr-Cys-Phe-Arg-Asn-Val-Gln-Asp-Asn | (SEQ ID N0: 1) |
| -Cys-Cys-Leu-Arg-Pro-Leu-Tyr-Ile-Asp-Phe-Lys-Arg-Asp- | |
| Leu-Gly | |

including any functional equivalents thereof obtained by addition, substitution or deletion of at least one amino acid.

In a further embodiment the immunogenic composition comprises a fragment of TGF-^{β} consisting of the amino acid sequence

| | |
|---|---|
| Ala-Leu-Asp-Ala-Ala-Tyr-Cys-Phe-Arg-Asn-Val-Gln-Asp-Asn | (SEQ ID NO: 1) |
| -Cys-Cys-Leu-Arg-Pro-Leu-Tyr-Ile-Asp-Phe-Lys-Arg-Asp- | |
| Leu-Gly | |

including any functional equivalents thereof obtained by addition, substitution or deletion of at least one amino acid.

In yet another embodiment there is provided an immunogenic composition comprising a fragment of TGF-^{β} essentially consisting of the amino acid sequence

| | |
|---|---|
| Ala-Leu-Asp-Ala-Ala-Tyr-Cys-Phe-Arg-Asn-Val-Gln-Asp-Asn | (SEQ ID NO: 1) |
| -Cys-Cys-Leu-Arg-Pro-Leu-Tyr-Ile-Asp-Phe-Lys-Arg-Asp- | |
| Leu-Gly. | |

When the amino acid sequence comprises a substitution of one amino acid for another, such a substitution may be a conservative amino acid substitution as defined herein above. Fragments of TGF-^{β} according to the present invention may comprise more than one such substitution, such as e.g. two conservative amino acid substitutions, for example three or four conservative amino acid substitutions, such as five or six conservative amino acid substitutions, for example seven or eight conservative amino acid substitutions, such as from 10 to 15 conservative amino acid substitutions, for example from 15 to 25 conservative amino acid substitution. Substitutions can be made within any one or more groups of predetermined amino acids as listed herein above under the section “Definitions”.

Examples of fragments comprising one or more conservative amino acid substitutions including one or more conservative amino acid substitutions within the same group of predetermined amino acids, or a plurality of conservative amino acid substitutions, wherein each conservative substitution is generated by substitution within a different group of predetermined amino acids as listed herein above, are listed herein below.

Accordingly, fragments of TGF-^{β} according to the invention may comprise within the same fragment or among different fragments, at least one substitution, such as a plurality of substitutions introduced independently of one another. Fragments may thus comprise conservative substitutions independently of one another, wherein at least one glycine (Gly) of said fragment of TGF-^{β} is substituted with an amino acid selected from the group of amino acids consisting of Ala, Val, Leu, and Ile, and independently thereof, fragments wherein at least one of said alanines (Ala) of said fragment of TGF-^{β} is substituted with an amino acid selected from the group of amino acids consisting of Gly, Val, Leu, and Ile, and independently thereof, fragments wherein at least one valine (Val) of said fragment of TGF-^{β} is substituted with an amino acid selected from the group of amino acids consisting of Gly, Ala, Leu, and Ile, and independently thereof, fragments wherein at least one of said leucines (Leu) of said fragment of TGF-^{β} is substituted with an amino acid selected from the group of amino acids consisting of Gly, Ala, Val, and Ile, and independently thereof, fragments wherein at least one isoleucine (Ile) of said fragment of TGF-^{β} is substituted with an amino acid selected from the group of amino acids consisting of Gly, Ala, Val and Leu, and independently thereof, fragments wherein at least one of said aspartic acids (Asp) of said fragment of TGF-^{β} is substituted with an amino acid selected from the group of amino acids consisting of Glu, Asn, and Gin, and independently thereof, fragments wherein at least one of said phenylalanines (Phe) of said fragment of TGF-^{β} is substituted with an amino acid selected from the group of amino acids consisting of Tyr, Trp, His, Pro, and preferably selected from the group of amino acids consisting of Tyr and Trp, and independently thereof, fragments wherein at least one of said tyrosines (Tyr) of said fragment of TGF-^{β} is substituted with an amino acid selected from the group of amino acids consisting of Phe, Trp, His, Pro, preferably an amino acid selected from the group of amino acids consisting of Phe and Trp, and independently thereof, fragments wherein at least one of said arginines (Arg) of said fragment of TGF-^{β} is substituted with an amino acid selected from the group of amino acids consisting of Lys and His, and independently thereof, fragments wherein at least one lysine (Lys) of said fragment of TGF-^{β} is substituted with an amino acid selected from the group of amino acids consisting of Arg and His, and independently thereof, fragments wherein at least one of said asparagines (Asn) of said fragment of TGF-^{β} is substituted with an amino acid selected from the group of amino acids consisting of Asp, Glut and Gin, and independently thereof, fragments wherein at least one glutamine (Gin) of said fragment of TGF-^{β} is substituted with an amino acid selected from the group of amino acids consisting of Asp, Glu, and Asn, and independently thereof, fragments wherein at least one proline (Pro) of said fragment of TGF-^{β} is substituted with an amino acid selected from the group of amino acids consisting of Phe, Tyr, Trp, and His, and independently thereof, fragments wherein at least one of said cysteines (Cys) of said fragment of TGF-^{β} is substituted with an amino acid selected from the group of amino acids consisting of Asp, Glu, Lys, Arg, His, Asn, Gin, Ser, Thr, and Tyr.

It is clear from the above outline that the same fragment may comprise more than one conservative amino acid substitution from more than one group of conservative amino acids as defined herein above.

It may also be possible to protect the fragments according to the invention against rearrangements. One such example of protection is the derivative Fmoc-Asp (Ompe) known to the skilled person and available for reducing and/or eliminating rearrangements caused by or involving Asp.

The addition or deletion of an amino acid may be an addition or deletion of from 2 to preferably 10 amino acids, such as from 2 to 8 amino acids, for example from 2 to 6 amino acids, such as from 2 to 4 amino acids. However, additions of more than 10 amino acids, such as additions from 10 to 200 amino acids, are also comprised within the present invention.

Specific examples of deletions comprise or essentially consist of residues 1 to 26, residues 1 to 23, and residues 1 to 20, respectively, of the fragment: Ala-Leu-Asp-Ala-Ala-Tyr-Cys-Phe-Arg-Asn-Val-Gln-Asp-Asn-Cys-Cys-Leu-Arg-Pro-Leu-Tyr-Ile-Asp-Phe-Lys-Arg-Asp-Leu-Gly; i.e. fragments or compositions of fragments comprising either

Ala-Leu-Asp-Ala-Ala-Tyr-Cys-Phe-Arg-Asn-Val-Gin-Asp-Asn-Cys-Cys-Leu-Arg Pro-Leu-Tyr-Ile -Asp-Phe-Lys-Arg; and/or

Ala-Leu-Asp-Ala-Ala-Tyr-Cys-Phe-Arg-Asn-Val-Gln-Asp-Asn-Cys-Cys-Leu-Arg-Pro-Leu-Tyr-Ile-Asp; and/or

Ala-Leu-Asp-Ala-Ala-Tyr-Cys-Phe-Arg-Asn-Val-Gln-Asp-Asn-Cys-Cys-Leu-Arg-Pro-Leu; including any combinations thereof.

Aditonal examples of specific deletions comprise or essentially consist of residues 4 to 29, residues 7 to 29, and residues 10 to 29. respectively, of the fragment: Ala-Leu-Asp-Ala-Ala-Tyr-Cys-Phe-Arg-Asn-Val-Gln-Asp-Asn-Cys-Cys-Leu-Arg-Pro-Leu-Tyr-Ile-Asp-Phe-Lys-Arg-Asp-Leu-Gly; i.e. fragments or compositions of fragments comprising either

Ala-Ala-Tyr-Cys-Phe-Arg-Asn-Val-Gln-Asp-Asn-Cys-Cys-Leu-Arg-Pro-Leu-Tyr-Ile-Asp-Phe-Lys-Arg-Asp-Leu-Gly; and/or

Cys-Phe-Arg-Asn-Val-Gin-Asp-Asn-Cys-Cys-Leu-Arg-Pro-Leu-Tyr-Ile-Asp-Phe-Lys-Arg-Asp-Leu-Gly, and/or

Asn-Val-Gin-Asp-Asn-Cys-Cys-Leu-Arg-Pro-Leu-Tyr-Ile-Asp-Phe-Lys-Arg-Asp-Leu-Gly; including any combinations thereof.

It will thus be understood that the invention pertains to immunogenic composition comprising at least one fragment of TGF-^{β} capable of eliciting an immunostimulating effect in an individual, including any variants and functional equivalents of such at least one fragment.

The fragment of TGF-^{β} according to the present invention, including any variants and functional equivalents thereof, may in one embodiment comprise less than 100 amino acid residues, such as less than 95 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues, such as less than 45 amino acid residues, for example less than 40 amino acid residues, such as less than 38 amino acid residues, for example less than 47 amino acid residues, such as less than 36 amino acid residues, for example less than 35 amino acid residues, such as less than 34 amino acid residues, for example less than 33 amino acid residues, such as less than 32 amino acid residues, for example less than 31 amino acid residues, such as about 30 amino acid residues, for example less than 30 amino acid residues, such as about 29 amino acid residues, for example 29 amino acid residues.

A fragment comprising the N-terminal 29 amino acids of mature TGF-^{β} is particularly preferred in one embodiment of the invention. However, the invention is not limited to fragments comprising at least 29 amino acids. Deletions of such fragments generating functionally equivalent fragments of TGF-P comprising less than 29 amino acids are also comprised in the present invention. Functionally equivalent TGF-^{β} peptides, and fragments thereof according to the present invention, may comprise less than 29 amino acid residues, for example less than 28 amino acid residues, such as less than 27 amino acid residues, for example less than 26 amino acid residues, such as less than 25 amino acid residues, for example less than 24 amino acid residues, such as less than 22 amino acid residues, for example less than 20 amino acid residues, such as less than 18 amino acid residues, for example less than 16 amino acid residues, such as less than 14 amino acid residues, for example less than 12 amino acid residues, such as less than 10 amino acid residues, for example less than 8 amino acid residues.

Functional equivalency as used in the present invention is according to one preferred embodiment established by means of reference to the corresponding functionality of a predetermined TGF-^{β} fragment, more preferably a fragment comprising the N-terminal 29 amino acids of mature TGF-^{β}, such as a fragment essentially consisting of the N-terminal 29 amino acids of mature TGF-^{β}, for example a fragment essentially consisting of the amino acid sequence Ala-Leu-Asp-Ala-Ala-Tyr-Cys-Phe-Arg-Asn-Val-Gln-Asp-Asn-Cys-Cys-Leu-Arg-Pro-Leu-Tyr-Ile-Asp-Phe-Lys-Arg-Asp-Leu-Gly (SEQ ID NO: 1)

Functional equivalents of a fragment of TGF-^{β} comprising a predetermined amino acid sequence are defined as stated herein above. One method of determining a sequence of immunogenically active amino acids within a known amino acid sequence has been described by Geysen in U.S. Pat. No. 5,595,915 and is incorporated herein by reference.

A further suitably adaptable method for determining structure and function relationships of peptide fragments is described by U.S. Pat. No. 6,013,478, which is herein incorporated by reference. Also, methods of assaying the binding of an amino acid sequence to a receptor moiety are known to the skilled artisan.

Functional equivalents of fragments of TGF-^{β} will be understood to exhibit amino acid sequences gradually departing from the preferred predetermined sequence Ala-Leu-Asp-Ala-Ala-Tyr-Cys-Phe-Arg-Asn-Val-Gin-Asp-Asn-Cys-Cys-Leu-Arg-Pro-Leu-Tyr-Ile-Asp-Phe-Lys-Arg-Asp-Leu-Gly (SEQ ID NO:. 1), as the number and scope of insertions, deletions and substitutions including conservative substitutions increases. This departure is measured as a reduction in homology between the preferred predetermined sequence and the variant or functional equivalent.

All immunostimulating TGF-^{β} fragments are included within the scope of this invention, regardless of the degree of homology that they show to a preferred predetermined sequence of TGF-^{β}. The reason for this is that some regions of TGF-^{β} are most likely readily mutatable, or capable of being completely deleted, without any significant effect on the immunostimulating activity of the resulting fragment.

A functional variant obtained by substitution may well exhibit some form or degree of native TGF-^{β} activity, and yet be less homologous if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity between i) a given TGF-^{β} fragment capable of eliciting an immunostimulating effect and ii) a preferred predetermined fragment, is not a principal measure of the fragment as a variant or functional equivalent of a preferred predetermined TGF-^{β} fragment according to the present invention.

Fragments sharing at least some homology with a preferred predetermined TGF-^{β} fragment of at least 29 amino acids are to be considered as falling within the scope of the present invention when they are at least about 40 percent homologous with the preferred predetermined TGF-^{β} fragment, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous homologous with the preferred predetermined TGF-^{β} fragment.

The homology between amino acid sequences may be calculated using well known algorithms such as for example any one of BLOSUM 30, BLOSUM 40, BLOSUM 45, BLOSUM 50, BLOSUM 55, BLOSUM 60 BLOSUM 62, BLOSUM 65, BLOSUM 70, BLOSUM 75, BLOSUM 80, BLOSUM 85, and BLOSUM 90.

Additional factors that may be taken into consideration when determining functional equivalence according to the meaning used herein are i) the ability of antisera which are capable of substantially neutralizing the growth inhibitory or the anchorage independent growth promoting activity of mature TGF-P to detect a TGF-P fragment according to the present invention, or ii) the ability of the functionally equivalent fragment to compete with TGF-^{β} for a cell surface receptor.

Conservative substitutions may be introduced in any position of a preferred predetermined fragment of TGF-^{β}. It may however also be desirable to introduce non-conservative substitutions, particularly, but not limited to, a non conservative substitution in, any one or more of the positions Arg18, Lys19, Leu20, Tyr21, Ile22, Phe24, Leu28, Gly29, Trp30, Trp32, Ile33, Pro36, Gly38, Tyr39, Asn42, Gly46, Pro49, Leu 62, Tyr65, Pro70, Val79, Pro80, Leu83, Leu86, Ile89, Val90, Tyr91, Tyr 92, Leu102, Asn105, Met106, Ile107 and Val108.

A non-conservative substitution leading to the formation of a functionally equivalent fragment of TGF-^{β} would for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gln, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

In a further embodiment the present invention relates to functional equivalents of a preferred predetermined fragment of TGF-^{β}, wherein such equivalents comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The importance of the hydrophilic and hydropathic amino acid indices in conferring interactive biologic function on a protein is well understood in the art (Kyte & Doolittle, 1982 and Hopp, U.S. Pat No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4 ); threonine (−0.7); serine (−0.8 ); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3);.valine (−1.5); leucine (−1.8); isoleucine (−1.8); tyrosine (−2.3); phenylalanine (−2.5); tryptophan (−3.4) (U.S Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In addition to the peptidyl compounds described herein, sterically similar compounds may be formulated to mimic the key portions of the peptide structure and that such compounds may also be used in the same manner as the peptides of the invention.

This may be achieved by techniques of modelling and chemical designing known to those of skill in the art. For example, esterification and other alkylations may be employed to modify, the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates formed with the same or other TGF-^{β} fragments and/or TGF-P molecules, including dimers or unrelated chemical moieties. Such functional equivalents are prepared by linkage of functionalities to groups which are found in fragment including at any one or both of the N- and C-termini, by means known in the art.

Functional equivalents may thus comprise fragments conjugated to aliphatic or acyl esters or amides of the carboxyl terminus, alkylamines or residues containing carboxyl side chains, e.g., conjugates to alkylamines at aspartic acid residues; O-acyl derivatives of hydroxyl group-containing residues and N-acyl derivatives of the amino terminal amino acid or amino-group containing residues, e.g. conjugates with fMet-Leu-Phe or immunogenic proteins. Derivatives of the acyl groups are selected from the group of alkyl-moieties (including C3 to C10 normal alkyl), thereby forming alkanoyl species, and carbocyclic or heterocyclic compounds, thereby forming aroyl species. The reactive groups preferably are difunctional compounds known per se for use in cross-linking proteins to insoluble matrices through reactive side groups.

Covalent or aggregative functional equivalents and derivatives thereof are useful as reagents in immunoassays or for affinity purification procedures. For example, a fragment of TGF-^{β} according to the present invention may be insolubilized by covalent bonding to cyanogen bromide-activated Sepharose by methods known per se or adsorbed to polyolefin surfaces either with or without glutaraldehyde cross-linking, for use in an assay or purification of anti-TGF-^{β} antibodies or cell surface receptors. Fragments may also be labelled with a detectable group, e.g., radioiodinated by the chloramine T procedure, covalently bound to rare earth chelates or conjugated to another fluorescent moiety for use in e.g. diagnostic assays.

Mutagenesis of a preferred predetermined fragment of TGF-^{β} can be conducted by making amino acid insertions, usually on the order of about from 1 to 10 amino acid residues, preferably from about 1 to 5 amino acid residues, or deletions of from about from 1 to 10 residues, such as from about 2 to 5 residues.

In one embodiment the fragment of TGF-^{β} is synthesised by automated synthesis. Any of the commercially available solid-phase techniques may be employed, such as the Merrifield solid phase synthesis method, in which amino acids are sequentially added to a growing amino acid chain. (See Merrifield, J. Am. Chem. Soc. 85:2149-2146, 1963). Equipment for automated synthesis of polypeptides is commercially available from suppliers such as Applied Biosystems, Inc. of Foster City, Calif., and may generally be operated according to the manufacturer's instructions. Solid phase synthesis will enable the incorporation of desirable amino acid substitutions into any fragment of TGF-^{β} according to the present invention. It will be understood that substitutions, deletions, insertions or any subcombination thereof may be combined to arrive at a final sequence of a functional equivalent. Insertions shall be understood to include amino-terminal and/or carboxyl-terminal fusions, e.g. with a hydrophobic or immunogenic protein or a carrier such as any polypeptide or scaffold structure capable as serving as a carrier.

Oligomers including dimers including homodimers and heterodimers of fragments of TGF-^{β} according to the invention are also provided and fall under the scope of the invention TGF-^{β} functional equivalents and variants can be produced as homodimers or heterodimers with other amino acid sequences or with native TGF-^{β} sequences. Heterodimers include dimers containing a TGF-^{β} fragment eliciting an immunostimulating effect when present in a homodimer, and a TGF-^{β} fragment that need not have or exert any biologically activity.

Immunostimulating TGF-^{β} fragments according to the invention may be synthesised both in vitro and in vivo. Method for in vitro synthesis are well known, and methods being suitable or suitably adaptable to the synthesis in vivo of TGF-^{β} are also described in the prior art. When synthesized in vivo, a host cell is transformed with vectors containing DNA encoding the TGF-^{β} fragment. A vector is defined as a replicable nucleic acid construct. Vectors are used to mediate expression of the TGF-^{β} fragment. An expression vector is a replicable DNA construct in which a nucleic acid sequence encoding the predetermined TGF-^{β} fragment, or any functional equivalent thereof that can be expressed in viva, operably linked to suitable control sequences capable of effecting the expression of the fragment or equivalent in a suitable host. Such control sequences are we known in the art.

Cultures of cells derived from multicellular organisms represent preferred host cells. In principle, any higher eukaryotic cell culture is workable, whether from vertebrate or invertebrate culture. Examples of useful host cell lines are VERO and HeLa cells, Chinese hamster ovary (CHO) cell lines, and W138, BHK, COS-7, 293 and MDCK cell lines. Preferred host cells are eukaryotic cells known to synthesize endogenous TGF-^{β}. Cultures of such host cells may be isolated and used as a source of the fragment, or used in therapeutic methods of treatment, including therapeutic methods aimed at promoting an immunostimulating effect, or diagnostic methods carried out an the human or animal body.

In another interesting embodiment of the present invention the fragment of TGF-^{β} capable of eliciting an immunostimulating effect comprises the amino acid sequence:

X-A-Arg-B-Leu-Tyr-Ile-Asp-Phe-H-I-Asp-Leu-Gly-Trp-Lys,

wherein X is Cys or a crosslinker moiety or a polypeptide that has at its C-terminus a Cys, and that, if greater than 15 residues does not have the sequence of mature or precursor TGF-^{β} at a homologous location in the mature or precursor TGF-^{β} molecule; and

wherein A is Val or Leu; B is Pro or Gln; H is Arg or Lys; and I is Lys, Arg, or Gln; or a physiologically acceptable salt or ester thereof; with the proviso that the TGF-^{β} fragment excludes (a) a full-length mature TGF-^{β} molecule or precursor TGF-^{β} molecule or deletion variants of mature or precursor TGF-^{β} molecules in which from about 1 to 10 amino acid residues have been deleted, (b) a fragment of the sequency: Cys-Val-Arg-Gin-Leu-Tyr-Ile-Asp-Phe-Arg-Lys-Asp-Leu-Gly-Trp-Lys, and (c) a fragment of the sequence: Arg-Asn-Leu-Glu-Glu-Asn-Cys-Cys-Val-Arg-Pro-Leu-Tyr-Ile-Asp-Phe-Arg-Gln-Asp-Leu.

Preferred fragments are Cys-Leu-Arg-Pro-Leu-Tyr-Ile-Asp-Phe-Lys-Arg-Asp-Leu-Gly-Trp-Lys (SEQ ID NO: 2); Cys-A-Arg-B-Leu-Tyr-Ile-Asp-Phe-H-I-Asp-Leu-Gly-Trp-Lys, and Cys-Val-Arg-B-Leu-Tyr-Ile-Asp-Phe-Arg-I-Asp-Leu-Gly-Trp-Lys, wherein: B is Pro or Gin; and/or I is Lys or Gin; and A and H are as defined herein above. It is more preferred that B is Gin and I is Lys, and that B is Pro and I is Gin.

The immunostimulating effect exerted by the TGF-^{β} fragments according to the present invention may be characterised by any one or a combination of i) a cytotoxic response and ii) an antibody response.

In one embodiment the TGF-^{β} fragments according to the present invention are themselves immunogenic. A TGF-^{β} fragment is considered to be immunogenic, when following immunisation of an individual with said TGF-^{β} fragment and an immunogenic determinant there is a detectable increase in the titer of antibodies against said. TGF-^{β} fragment in the serum of said individual. Such increase in antibody titer can be measured by any suitable method known to the person skilled in the art. One example of such a method is and Elisa assay. However, for the purposes of the present invention, it is not required that the TGF-^{β} fragment is immunogenic. Hence, the immunostimulating fragments of TGF-^{β} according to the present invention may be either immunogenic or they may not be immunogenic.

The immunogenic composition according to the invention may generate an enhanced immune response caused by an enhanced increase in at least one class of immunoglobulins and preferably an enhanced increase in a plurality of immunoglobulin classes such as more than one class including two classes, such as three classes, for example four classes of immunoglobulins. The response may also be caused by an increase in the level of T-cells, such as an increase in the level of cytotoxic T-cells, or by an increase in the level of at least one immunoglobulin class as well as an increase in the level of T-cells.

The enhanced immune response achieved according to the invention can be attributable to e.g. an enhanced increase in the level of immunoglobulins or in the level of T-cells including cytotoxic T-cells will result in immunisation of at least 50% of individuals exposed to said immunogenic composition or vaccine, such as at least 55%, for example at least 60%, such as at least 65%, for example at least 70%, for example at least 75%, such as at least 80%, for example at least 85%, such as at least 90%, for example at least 92%, such as at least 94%, for example at least 96%, such as at least 97%, for example at least 98%, such as at least 98.5%, for example at least 99%, for example at least 99.5% of the individuals exposed to said immunogenic composition or vaccine are immunised.

Compositions according to the invention may also comprise any carrier and/or adjuvant known in the art including functional equivalents thereof. Functionally equivalent carriers are capable of presenting the same immunogenic determinant in essentially the same steric conformation when used under similar conditions. Functionally equivalent adjuvants are capable of providing similar increases in the efficacy of the composition when used under similar conditions.

Preferably, said compositions comprise potent, nontoxic adjuvants that will enhance and/or modulate the immunogenicity of immunogenic determinants including antigenic determinants including haptenic determinants represent one group of preferred adjuvants. In addition, such adjuvants preferably also elicit an earlier, more potent, or more prolonged immune response. Such an adjuvant would also be useful in cases where an antigen supply is limited or is costly to produce.

Adjuvants pertaining to the present invention may be grouped according to their origin, be it mineral, bacterial, plant, synthetic, or host product The first group under this classification is the mineral adjuvants, such as aluminum compounds. Antigens precipitated with aluminum salts or antigens mixed with or adsorbed to performed aluminum compounds have been used extensively to augment immune responses in animals and humans. Aluminium particles have been demonstrated in regional lymph nodes of rabbits seven days following immunisation, and it may be that another significant function is to direct antigen to T cell containing areas in the nodes themselves. Adjuvant potency has been shown to correlate with intimation of the draining lymph nodes, While many studies have confirmed that antigens administered with aluminium salts lead to increased humoral immunity, cell mediated immunity appears to be only slightly increased, as measured by delayed-type hypersensitivity. Aluminium hydroxide has also been described as activating the complement pathway. This mechanism may play a role in the local inflammatory response as well as immunoglobulin production and B cell memory. Furthermore, aluminum hydroxide can protect the antigen from rapid catabolism. Primarily because of their excellent record of safety, aluminum compounds are presently the only adjuvants used in humans.

While aluminum salts have been a sufficient adjuvant for strong immunogens that require only antibody responses to elicit protection, they may not always be effective when used with weak immunogens such as e.g. synthetic peptides of malaria, or for introducing cell-mediated immune responses or IgG isotype of the type required to fight infections. Thus, the immunostimulating fragment of TGF-^{β} according to the present invention may in one embodiment act as an adjuvant or immunostimulator and may be conjugated or non-conjugated to the immunogenic determinant against which it is desirable to raise an immune response.

Another large group of adjuvants is those of bacterial origin. Adjuvants with bacterial origins can be purified and synthesized (e.g. muramyl dipeptides, lipid A) and host mediators have been cloned (interleukin 1 and 2). The last decade has brought significant progress in the chemical purification of several adjuvants of active components of bacterial origin: Bordetella pertussis, Mycobacterium tuberculosis, lipopolysaccharide, Freund's Complete Adjuvant (FCA) and Freund's Incomplete Adjuvant (Difco Laboratories, Detroit, Mich.) and Merck Adjuvant 65 (Merck and Company, Inc., Rahway, N.J.). Additionally suitable adjuvants in accordance with the present invention are e.g. Titermax Classical adjuvant (SIGMA-ALDRICH). ISCOMS, Quil A, ALUN, see U.S. Pat. Nos. 58767 and 5,654,372, Lipid A derivatives, choleratoxin derivatives, HSP derivatives, LPS derivatives, synthetic peptide matrixes, GMDP, and other as well as combined with immunostimulants (U.S. Pat. No. 5,876,735).

B. pertussis is of interest as an adjuvant in the context of the present invention due to its ability to modulate cell-mediated immunity through action on T-lymphocyte populations. For lipopolysaccharide and Freund's Complete Adjuvant, adjuvant active moieties have been identified and synthesized which permit study of structure-function relationships. These are also considered for inclusion in immunogenic compositions according to the present invention.

Lipopolysaccharide and its various derivatives, including lipid A, have been found to be powerful adjuvants in combination with liposomes or other lipid emulsions. It is not yet certain whether derivatives with sufficiently low toxicity for general use in humans can be produced. Freund's Complete Adjuvant is the standard in most experimental studies.

Mineral oil may be added to vaccine formulation, in order to protect the antigen from rapid catabolism.

Many other types of materials can be used as adjuvants in immunogenic compositions according to the present invention. They include plant products such as saponin, animal products such as chitin and numerous synthetic chemicals.

Adjuvants according to the present invention can also been categorized by their proposed mechanisms of action. This type of classification is necessarily somewhat arbitrary because most adjuvants appear to function by more than one mechanism. Adjuvants may act through antigen localization: and delivery, or by direct effects on cells making up the immune system, such as macrophages and lymphocytes. Another mechanism by which adjuvants according to the invention enhance the immune response is by creation of an antigen depot. This appears to contribute to the adjuvant activity of aluminum compounds, oil emulsions, liposomes, and synthetic polymers. The adjuvant activity of lipopolysaccharides and muramyl dipeptides appears to be mainly mediated through activation of the macrophage, whereas B. pertussis affects both macrophages and lymphocytes. Further examples of adjuvants that may be useful when incorporated into immunogenic compositions according to the present invention are described in U.S. Pat. No. 5,554,372.

In one preferred embodiment, adjuvants according to the present invention are selected from the group consisting of aluminium compounds, Freunds incomplete adjuvant, Titermax classical adjuvant and oil emulsions.

There is also provided an embodiment of the present invention wherein the immunogenic composition further comprises a carrier. The carrier may be present independently of an adjuvant. The purpose of conjugation and/or co-immunisation of an immunogenic determinant and a carrier can be e.g to increase the molecular weight of the immunogenic determinant in order to increase the activity or immunogenicity of the determinant, to confer stability to the determinant, to increase the biological activity of the determinant or to increase its serum half-life. The carrier protein may be any conventional carrier including any protein suitable for presenting immunogenic determinants. Conventional carrier proteins include, but are not limited to, keyhole limpet hemocyanin, serum proteins such as transferrin, bovine serum albumin, or human serum albumin, an ovalbumin, immunoglobulins, or hormones, such as insulin.

While any suitable pharmaceutical carrier known to those of ordinary skill in the art may be employed in the immunogenic and pharmaceutical compositions of this invention, the type of pharmaceutical carrier Will vary depending on the mode of administration and whether a sustained release administration is desired. For parenteral administration, such as subcutaneous injection, the pharmaceutical carrier may e.g. comprise water, saline, alcohol, fat, a wax or a buffer. For oral administration, any of the above pharmaceutical carriers or a solid pharmaceutical carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as pharmaceutical carriers for the pharmaceutical compositions of this invention. Suitable biodegradable microspheres are disclosed, for example, in U.S. Pat. Nos. 4,897,268 and 5,075,109.

The immunogenic determinant and/or the immunostimulating fragment of TGF-^{β} may be encapsulated within the biodegradable microsphere or associated with the surface of the microsphere.

Immunogenic compositions, pharmaceutical compositions and vaccines according to the invention may also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with non-specific serum albumin are exemplary appropriate diluents. Preferably, the compostions are formulated as a lyophilizate using appropriate excipient solutions (e.g., sucrose) as diluents.

The immunogenic composition may comprise an immunogenic determinant including an antigenic determinant including a hapten that is either conjugated or non-conjugated, and—independently thereof—an immunostimulating fragment of TGF-^{β} that is either conjugated or non-conjugated. Accordingly, a number of compositions comprising conceivable combinations of conjugated and/or non-conjugated immunogenic determinants and conjugated and/or non-conjugated immunostimulating fragments of TGF-^{β} according to the present invention are listed herein below.

Compounds within the scope of the present invention could be conjugated by any method known to the person skilled in the art. For example they could be conjugated by a physical association generated for example by the formation of a chemical bond, such as for example a covalent bond, formed between compounds to be conjugated. Compounds could be conjugated for example by an oxidative induced cross-link, such as mild oxidative induced cross-link catalysed by long-time exposure to atmospheric air, such as an over-night exposure. Alternatively, compounds could be conjugated using a chemical cross-linking reagent. Examples of chemical cross-linking reagents are glutaraldehyde, charbodiimid or formaldehyde

The present invention in one embodiment provides any one or more of an immunogenic composition wherein said immunostimulating fragment of TGF-^{β} and said immunogenic determinant are both non-conjugated, an immunogenic composition wherein said fragment is conjugated and said immunogenic determinant is non-conjugated, an immunogenic composition wherein said fragment is non-conjugated and said immunogenic determinant is conjugated, and an immunogenic composition wherein said fragment and said immunogenic determinant are both conjugated, including a composition wherein said fragment is conjugated to said immunogenic determinant.

The invention also provides any one or more of an immunogenic composition further comprising a carrier, wherein said fragment and said immunogenic determinant are both non-conjugated, an immunogenic composition comprising a carrier wherein said fragment and said immunogenic determinant are both conjugated, an immunogenic composition comprising a carrier wherein said carrier is non-conjugated, an immunogenic composition comprising a carrier wherein said carrier is conjugated, an immunogenic composition comprising a carrier wherein said fragment is conjugated and said immunogenic determinant is non-conjugated, an immunogenic composition comprising a carrier wherein said fragment is non-conjugated and said immunogenic determinant is conjugated

When the immunogenic composition comprises a fragment that is conjugated and an immunogenic determinant that is nonconjugated, the carrier may be either non-conjugated or conjugated, including conjugated to said fragment

When the immunogenic composition comprises a fragment that is non-conjugated and an immunogenic determinant that is conjugated, the carrier may be either non-conjugated or conjugated, including conjugated to said immunogenic determinant.

When the immunogenic composition comprises a fragment and an immunogenic determinant that are both conjugated, including the embodiment wherein the fragment is conjugated to the immunogenic determinant, the carrier may be either non-conjugated or conjugated. When the carrier is conjugated, the fragment may be conjugated to said carrier, or the immunogenic determinant may be conjugated to said carrier, or the carrier may be conjugated to one or both of said fragment and said immunogenic determinant.

The immunogenic composition may—in addition to a carrier—further comprise an adjuvant as described herein above. This may be the case e.g. when the invention pertains to a vaccine comprising an immunogenic composition according to the invention. Any adjuvant can be used in combination with the compositions according to the present invention.

In a further embodiment there is provided an immunogenic composition or a vaccine according to the invention for use in a method of immunising an individual in need of immunisation. The method of immunising an individual in need of immunisation comprises the steps of

providing an immunogenic composition or a vaccine according to the invention, and

administering said immunogenic composition or said vaccine to said individual.

It is preferred that at least 50% of individuals administered said immunogenic composition or said vaccine are immunised, such as at least 55%, for example at least 60%, such as at least 65%, for example at least 70%, for example at least 75%, such as at least 80%, for example at least 85%, such as at least 90%, for example at least 92%, such as at least 40%, for example at least 96%, such as at least 97%, for example at least 98%, suck as at least 98.5%, for example at least 99%, for example at least 99.5%, such as at least 99.9%, for example at least 99.99% of the individuals administered said immunogenic composition or said vaccine are immunised.

The individual in need of immunisation could be any individual susceptible to infection by the agent against which is immunised. Preferably such an individual is a vertebrate, which could be any vertebrate. Examples of vertebrates are mammals including human beings and rodents, such as for example rabbits or fish for example rainbow trout (Oncorhynchus mykiss) or Atlantic salmon.

In further embodiments there are provided the use of a fragment of TGF-^{β} capable of facilitating an immunostimulating effect in an individual in the manufacture of an immunogenic composition or a vaccine, and the use of a fragment of TGF-^{β} for the manufacture of a medicament or enhancing the immunostimulating effect of an immunisation.

A “pharmacologic dosage” comprises an effective amount of an immunostimulating TGF-^{β} fragment or an effective amount of an immunological determinant, and provides in both cases a desired physiological effect. This amount may vary to some degree depending on the mode of administration, but the amount will normally be in the same general range, respectively, for the fragment and the determinant. If more than one immunostimulating fragment is used, each one may be present in these amounts or the total amount may fall within the ranges illustrated above.

The effective amount of the immunogenic fragment of TGF-^{β} comprised in the immunogenic compositions of the present invention will vary according to the individual in need of immunisation, the more of administration and the condition against which is immunised In general, when the composition is injected subcutaneously into rabbits, the amount is preferably between 0.5 and 500 ^{μ}g, more preferably between 1 and 200 ^{μ}g, yet more preferably between 4 and 100 μg. When injected intraperitonally into rainbow trouts the amount is preferably between 0.5 and 500 ^{μ}g, more preferably between 1 and 200 ^{μ}g, yet more preferably around 15 ^{μ}g. An effective amount of an immunogenic determinant may be an amount capable of eliciting a detectable humoral immune response in the absence of an immunomodulator. For many immunogens, this is in the range of about 5-100 ^{μ}g for a human subject. Since the vaccines of the invention utilize an immunostimulating fragment of TGF-^{β} capable of enhancing the humoral immune response, it may be possible to utilize a smaller amount of the immunogenic determinant, e.g., about 1-5 ^{μ}g for a human subject, The appropriate amount of immunogen to be used is dependent on the immunological response it is desired to elicit.

If injected the effective amount of immunogenic determinant will typically be in the range of from about 0.1 to about 1000 ^{μ}g, such as e.g. from about 1 to about 900 ^{μ}g, for example from about 5 to about 500 ^{μ}g, for a human subject, and generally in the range of from about 0.01 to 10.0 ^{μ}g/Kg body weight of a subject animal. The above-indicated ranges are merely indicative and should not be interpreted as limiting the present invention.

The exact effective amount necessary will vary from subject to subject, depending on the species, age and general condition of the subject, the severity of the condition being treated, the mode of administration, etc. It is therefore not always possible to specify an exact effective amount. However, the appropriate effective amount may be determined by one of ordinary skill in the art using only routine experimentation or prior knowledge in the art

The mode of administration of the immunogenic composition according to the present invention will depend on the individual to be immunised, the immunogenic determinant, the TGF-^{β} fragment and the presence of other components of the composition such as carriers and/or adjuvants. The below mentioned modes of administration are examples and should be regarded as limiting to the invention.

Modes of administration of the composition according to the invention include, but are not limited to, subcutaneous administration, intradermal administration, intramuscular administration, nasal administration, oral administration, and generally any form of mucosal administration. Preferably, such administration is by injection.

The administration of each unit dose could be at least once, such as at least twice, for example at least 3 times, such as at least 5 times, for example at least 10 times. There could be a time gap between two administration of at least 1 day, such as at least 2 days, for example at least 1 week, such as at least 2 weeks, for example at least one month, such as at least 2 months, for example at least 6 months, such as at least one year. The time gap between administration could be the same amount of time between any administrations, or it could differ from time to time. In one embodiment the adminstration is 3 times with a time gap of around 2 weeks between every administration.

The volume of each unit dose will vary upon a number of factors as mentioned above. Preferbaly, when immunising rabbits by injection the volume is between 0.05 and 1.0 ml, more preferably between 0.1 and 0. 2 ml.

The immunogenic determinant and the fragment of TGF-^{β} according to the present invention may be administrated simultaneously either comprised within a single formulation or in separate formulations or they may be administered sequentially.

The immunostimulating effect according to the present invention can be determined in vivo, by measuring e.g. an increased T cell responsiveness to T cell dependent immunogenic determinants, wherein said increased responsiveness is characteristic of an enhancement of a normal immune response to such antigens. An immunostimulating effect may also be measured as an enhanced T cell production of, in particulars IL-2, IL-3, IFN-^{γ} and/or GM-CSF. Fragments of TGF-^{β} with a potential for eliciting an enhanced immune response may thus be readily identified by screening for enhanced IL-2, IL-3, IFN-^{γ} or GM-CSF production by T cells, as described e.g. in U.S. Ser. No. 07/779,499, incorporated herein by reference.

An immunostimulating effect shall also be understood to comprise any effect exerted by immunogenic compositions according to the present invention when they are administered to an individual including a vertebrate animal in vivo at a peripheral site, of altering the local environment of a peripheral lymphoid organ that drains from the administration site, such that activated lymphocytes and macrophages residing within the lymphoid organ exhibit a pattern of cytokines more typical of the local environment of a lymphoid organ of the mucosal lymphoid compartment. Particularly, a pattern of cytokines more typical of a mucosal lymphoid organ is characterized by relatively enhanced production of one or more of active TGF-^{β}, IL-4, IL-5, and IL-10, and a decreased production (or at least no relatively enhanced production) of one or more of IL-2 and IFN-^{γ}.

The vaccine chip technology according to the present invention makes it possible to improve conventional vaccines comprising at least one immunogenic determinant against which it is desirable to elicit an immune response. The enhanced immunogenicity exerted by the antigen determinant in question sides in the immunostimulating effect provided by the fragment of TGF-^{β} that is included in the vaccine chip technology. As examples of conventional vaccines capable of being improved by inclusion of an immunostimulating fragment of TGF-^{β} according to the present invention are e.g. conventional vaccines aiming to provide a protective immune response against e.g. Actinomycosis, Adenovirus-infections, Antrax, Bacterial dysentery, Botulisme, Brucellosis (Bang's disease), preferably caused by B. melitensis and B. suis, Candidasis, Cellulitis, Chancroid, Cholera, Coccidioidomycosis, Acute afebril, Conjunctivitis, Cystitis, Dermatophytosis, Difteri, Bacterial Endocarditis, Epiglottitis, Erysipelas, Erysipeloid, Gastroenteritis, Genital herpes, Glandulae, Gonorrhea, Viral Hepatitis, Histoplasmose, Impetigo, Mononucleosis, Influenza, Legionaires disease, Spedalskhed, Leptospirosis, Lyme disease, Melloidosis, Meningitis, Faresyge, Nocardiosis Nocardia asteroides, Non-gonococcal urethritis, Pinta, Pest, Pneumococcal lungebetændelse, Poliomyelitis, Primary lung infection, Pseudomembranøs enterocolitis, antibiotic-associated Puerperal sepsis, Rabies, Relaps-fever, Rheumatic fever, Rocky Mountain spotted-fever, Rubella, Rubeola, Nældefever, Staphylococcal scalded skin syndrome, Streptococcal pharyngitis (strep throat), Syfilis, Tetanus, Toxic shock syndrome, Toxoplasmose, Tuberculosis, Tularemia, Typhoid fever, Tyfus, Vaginitis, Varicella, Verrucae, Pertussis, Framboesia (Yaws) and Yellow fever.

Additional examples of conventional vaccines capable of benefiting from the present invention are each and everyone of the four below-mentioned classes of vaccine which have been developed against mammalian diseases, and all four classes are capable of being supplemented with immunostimulating fragments of TGF-^{β} according to the present invention. The four major classes include: Live-attenuated vaccines; non-living whole vaccines; vector vaccines including DNA or RNA vaccines; and subunit vaccines. Several reviews discuss the preparation and utility of these classes of vaccines. See for example, Subbarao et. al. (1992) in Genetically Engineered Vaccines, edited by Ciardi et al., Plenum Press, New York; and Melnick (1985) in High Technology Route to ,Virus Vaccines, edited by Dreesman et al., published by the American Society for Microbiology, the disclosures of which are incoporated herein by reference.

Live attenuated vaccines comprise live but attenuated, i.e., non-virulent, pathogens that have been “crippled” by means of genetic mutations. The mutations prevent the pathogens from causing disease in the recipient or vaccinee. The primary advantage of this type of vaccine is that the attenuated organism stimulates the immune system of the recipient in the same manner as the wild type pathogen by mimicking the natural infection. Furthermore, the attenuated pathogens replicate in the vaccine thereby presenting a continuous supply of immunogenic determinants to the recipient's immune system. It is an aim of the present invention to improve the efficacy of a live attenuated vaccine developed against e.g smallpox; yellow fever; measles; mumps; rubella; poliomyelitis; adenovirus; and tuberculosis, with with at least one immunostimulating fragment of TGF-P according to the present invention.

Non-living whole vaccines comprise non viable whole organisms. The pathogens are routinely inactivated either by chemical treatment, i.e., formalin inactivation, or by treatment with lethal doses of radiation. It is an aim of the present invention to improve the efficacy of a non-living whole vaccine against e.g. pertussis; typhus; typhoid fever; paratyphoid fever; and particular strains of influenza, with with at least one immunostimulating fragment of TGF-P according to the present invention.

Vector vaccines, also known as live recombinant vehicle vaccines, are prepared by incorporating a gene encoding a specific immunogenic determinant of interest into a living but harmless virus or bacterium. The harmless vector organism is injected into the intended recipient In principle, the recombinant vector organism replicates in the host producing and presenting the immunogenic determinant to the host's immune system. It is contemplated that this type of vaccine will be more effective than the non-replicative type of vaccine. For such a vaccine to be successful, the vector must be viable, and either be naturally non-virulent or have an attenuated phenotype.

It is an aim of the present invention to improve the efficacy of vectors such as specific strains of vaccinia (cowpox) virus, adenovirus, adeno-associated virus, salmonella and mycobacteria. Improved live strains of vaccinia virus and mycobacteria that are capable of being administered safely to humans in the forms of the smallpox and tuberculosis (BCG) vaccines, respectively, are also claimed. They have been shown to express foreign proteins and exhibit little or no conversion into virulent phenotypes. It is a further aim of the present invention to improve the efficacy of any vector vaccine using the BCG vector, such as vector vaccines directed against the human immunodeficiency virus (HIV). For example, the HIV antigenic proteins: gag; env; HIV protease; reverse transcriptase; gp120 and gp41 have been introduced, one at a time, into the BCG vector and shown to induce T-cell mediated immune responses against the HIV proteins in animal models (Aldovini et al. (1991) Nature 351:479-482; Stover et al. (1991) Nature 351:456-460; Colston (1991) Nature 351:442-443).

Vector vaccines comprising an immunostimulating TGF-^{β} fragment according to the present invention and capable of carrying a plurality of foreign genes thereby permitting simultaneous vaccination against a variety of preselected immunogenic determinants are particularly preferred As an example, several HIV genes have been engineered into the vaccinia virus genome thereby creating multivalent vaccines which are, in theory, capable of simultanaeously stimulating a response against several HIV proteins.

Further examples of vector vaccines such as DNA or RNA vaccines pertain to the introduction of e.g. an immunogenic determinant into a patient by overexpressing in the cells of the patient a nucleic acid construct which includes expression control sequences operably linked to a sequence encoding the immunogenic determinant. As such peptides may not contain a methionine start codon, such a codon is optionally included as part of the expression control sequences. The nucleic acid construct may be a non-replicating linear or circular DNA or RNA vector, or an autonomously replicating plasmid or viral vector, or th construct may be integrated into the host genome. Any vector that can transfect a mammalian cell may be used in the methods of immunising an individual according to the present invention. Methods for constructing expression vectors are well known in the art (see, e.g., Molecular Cloning: A Laboratory Manual, Sambrook et al., eds., Cold Spring Harbor Laboratory, 2nd Edition, Cold Spring, Harbor, N.Y., 1989).

Further examples of vector vaccines capable of being improved by incorporation therein of an immunostimulating fragment of TGF-^{β} according to the present invention are vector vaccines comprising e.g., retroviruses, as disclosed in WO 90/07936, WO 91/02805, WO 93/25234, WO 93/25698, and WO 94/03622, adenovirus, as disclosed by Berkner, Biotechniques 6:616-627, 1988; Li et al., Hum. Gene Ther. 4:403-409, 1993; Vincent et al., Nat. Genet. 5:130-134, 1993; and Kolls et al., Proc. Natl. Acad. Sci. USA 91:215-219, 1994) pox virus, as disclosed by U.S. Pat. Nos. 4,769,330; 5,017,487; and WO 89/01973, naked DNA as disclosed WO 90/11092, a nucleic acid molecule complexed to a polycationic molecule as disclosed in WO 93/03709, and nucleic acids associated with liposomes as disclosed by Wang et al., Proc. Natl. Acad. Sci. USA 84:7851, 1987.

In certain embodiments, the DNA may be linked to killed or inactivated adenovirus as disclosed by Curiel et al., Hum. Gene Ther. 3:147-154, 1992; Cotton et al., Proc. Natl. Acad. Sci. USA 89;6094, 1992. Other suitable compositions include DNA-ligands as disclosed by Wu et al., J. Biol. Chem. 264:16985-16987, 1989), and lipid-DNA combinations as disclosed by Feigner et al., Proc. Natl. Acad. Sci USA 84:7413-7417, 1989). In addition, the efficiency of naked DNA uptake into cells may be increased by coating the DNA onto biodegradable latex beads.

In addition to direct in vivo procedures, ex vivo procedures may be used in which cells are removed from an animal, modified, and placed into the same or another animal. It will be evident that one can utilize any of the compositions noted above for introduction of an immunogenic determinant according to the invention into tissue cells in an ex vivo context. Protocols for viral, physical and chemical methods of uptake are well known in the art.

Vaccine vectors preferably comprise a suitable promoter is operably linked to the nucleic acid sequence encoding the immunogenic determinant. Any promoter that can direct a high level of transcription initiation in the target cells may be used in the invention. Non-tissue specific promoters, such as the cytomegalovirus (DeBernardi et al., Proc Natl Acad Sci USA 88:9257-9261 [1991], and references therein), mouse metallothionine I (Hammer et al., J Mol Appl Gen 1-273-288 [1982]), HSV thymidine kinase (McKnight, Cell 31:355-365 [1982]), and SV40 early (Benoist et al. Nature 290:304-310 [1981]) promoters may be used in methods of the invention, as over-expression of immunogenic determinants in the methods pertaining to the invention would be expected not to adversely affect transfected cells.

The above-described nucleic, acid constructs and vectors can be introduced into target cells in vivo or in vitro by any standard method, e.g., as naked DNA (Donnelly et al., Annu Rev Immunol 15:617-648 [1997]), incorporated into ISCOMS, liposomes, or erythrocyte ghosts, or by biolistic transfer, calcium precipitation, or electroporation. Alternatively, one can employ a viral-based vector as a means for introducing the nucleic acid encoding the immunogenic determinant into the cells of the animal. Preferred viral vectors include those derived from replication-defective hepatitis viruses (e.g, HBV and HCV), retroviruses (see, e.g., WO89/07136; and Rosenberg et al., N Eng J Med 323 (9):570-578 [1990]), adenovirus (see, e.g., Morsey et al., J Cell Biochem. Supp. 17E [1993]), adeno-associated virus (Kotin et al., Proc Natl Acad Sci USA 87:2211-2215 [1990]), replication defective herpes simplex viruses (HSV; Lu et al., Abstract, page 66, Abstracts of the Meeting on Gene Therapy, Sep. 22-26, 1992, Cold Spring Harbor Laboratory, Cold Spring Harbor, N.Y.), canary pox virus, and any modified versions of these vectors. Cells transfected in vitro can be cultured and cloned, if desired, prior to introduction into the patient.

As an alternative to administration of the immunogenic determinant or a vector capable of expressing such a determinant directly to the patient, one can remove helper T cells from the patient; stimulate those T cells ex vivo using the same immunogenic determinant or vector, and introduce the stimulated helper T cells into the same patient.

Another group of vaccines capable of benefitting from the present invention are subunit vaccines. These vaccines usually comprise a subcellular component purified from a pathogen against which it is desirable to immunise an individual. Subunit vaccines are usually safe to administer since it is unlikely that the subcellular components will cause disease in the recipient. The purified subcellular component may be either a defined subcellular fraction, purified protein, nucleic acid or polysaccharide having an immunogenic determinant capable of stimulating an immune response against the pathogen. The immunogenic components can be purified from a preparation of disrupted pathogens. Alternatively, the immunogenic proteins, nucleic acids or polysaccharides may be synthesized using procedures well known in the art. It is an aim of the present invention to improve the efficacy of subunit vaccines including, but not limited to, subunit vaccines against cholera; diphtheria; hepatitis type B; poliomyelitis; tetanus; and specific strains of influenza.

The immunogenic composition according to the invention may be administered subcutaneously according to well known techniques, or epicutaneously at a peripheral anatomical site (such as, for human subjects, for example, the arm or buttocks or leg); and the specific antigen is administered to the same anatomical site, or to a site known to drain into the same lymphoid organ that receives drainage from the site of administration of the composition. In one mode of administration, the immunostimulating TGF-^{β} fragment is combined with an immunogenic determinant for simultaneous administration at the same site.

It invention is also useful in connection with autoimmune therapy or autoimmune disease control in an individual. This would be the case when e.g. the immune system of an individual becomes unbalanced and the body begins to manufacture antibodies and T cells directed against the body's own constituents-cells, cell components, or specific organs. Such antibodies are known as autoantibodies, and the diseases they produce are called autoimmune diseases.

Autoimmune diseases affect the immune system at several levels. In patients with SLE, for instance, B cells are hyperactive while suppressor cells are underactive; it is not clear which defect comes first Moreover, production of IL-2 is low, while levels of gamma interferon are high. Patients with rheumatoid arthritis, who have a defective suppressor T cell system, continue to make antibodies to a common virus, whereas the response normally shuts down after about a dozen days. Autoantibodies to red blood cells can cause anemia, autoantibodies to pancreas cells may contribute to juvenile diabetes, and autoantibodies to nerve and muscle cells are found in patients with the chronic muscle weakness known as myasthenia gravis. Autoantibody known as rheumatoid factor is common in persons with rheumatoid arthritis. Persons with systemic lupus erythematosus (SLE), whose symptoms encompass many systems, have antibodies to many types of cells and cellular components. These include antibodies directed against substances found in the cell's nucleus-DNA, RNA, or proteins-which are known as antinuclear antibodies, or ANAs. These antibodies can cause serious damage when they link up with self antigens to form circulating immune complexes, which become lodged in body tissue and set off inflammatory reactions (Immune Complex Diseases). Accordingly, in one embodiment the immunogenic composition according to the invention comprises an immunostimulating fragment of TGF-^{β} and an immunogenic determinant capable of eliciting an immune response at least involving the synthesis of autoantibodies.

The vaccine chip technology according to the present invention is also useful for providing immunogenic compositions comprising an immunostimulating fragment of TGF-^{β} and an immunogenic determinant capable of exploitation in fields as diverse as cancer immunotherapy, immune castration, and immune contraception. In one such embodiment the immunogenic determinant is a self-antigen such as e.g. a self-antigen directly or indirectly involved in the development of a cancer. It is well known that the cells of the immune system can proliferate uncontrollably and result in the development of a cancer. Leukemias are caused by the proliferation of white blood cells, or leukocytes. The uncontrolled growth of antibody-producing (plasma) cells can lead to multiple myeloma. Cancers of the lymphoid organs, known as lymphomas, include Hodgkin's disease. Accordingly, immunogenic compositions according to the invention comprising an immunostimulating fragment of TGF-^{β} and an immunogenic determinant can be used for autoimmune disease control and/or for autoimmune cancer therapy by promoting an immune response against predetermined self-antigens.

Additionally, when the immunogenic determinant is a self-antigen directly or indirectly involved in the development of a cancer, such cancer could for example be uveal melanoma, malignant glioma, prostate cancer, skin cancer, liver cancer, breast cancer or colorectal cancer.

Furthermore, the vaccine chip technology according to the present invention is also useful for providing immunogenic compositions comprising an immunostimulating fragment of TGF-^{β} and an immunogenic determinant, which is related to astma.

The TGF-^{β} fragments according to the present invention may in one embodiment be used as vaccine adjuvants or in combination with known vaccine adjuvants to enhance a vaccine-induced humoral immune response. When an individual is immunized with an immunizing agent,, administration of the immunostimulating TGF-^{β} fragment may be prior to, contemporaneously with, or after the vaccination. Typical methods of administering the TGF-^{β} fragment include mixing the fragment with the immunogenic determinant in a vaccine or topically applying the TGF-^{β} fragment to skin sites which drain into the same lymph nodes as the immunogenic determinant comprised in the vaccine. This latter method is preferably used with individuals who are immunologically deficient and in whom one wishes to augment the immune response, for example, the aged or neonates, or individuals who are therapeutically immunosuppressed.

One or more immunostimulating fragments can be used to enhance the vaccine-induced immune response. They may be administered sequentially or contemporaneously. It is preferred to administer them contemporaneously and in a single vehicle.

Immunogenic determinants according to the present invention can be e.g. a protein, a polysaccharide, a lipopolysaccharide or a lipopeptide, or any part thereof; or it can be a combination of any of these including a combination of any parts thereof. Particularly, the specific immunogenic determinant can include a native protein or protein fragment, or a synthetic protein or protein fragment or peptide; it can include glycoprotein, glycopeptide, lipoprotein, lipopeptide, nucleoprotein, nucleopeptide; it can include a peptide-peptide conjugate, or it can include a recombinant nucleic acid expression product. The immunogenic determinant may further be a DNA molecule which produces an immunogen or an antigen in the vertebrate.

Examples of immunogenic determinants include, among others, those that are capable of eliciting an immune response against viral or bacterial hepatitis, influenza, diphtheria, tetanus, pertussis, measles, mumps, rubella, polio, pneumococcus, herpes, respiratory syncytial virus, haemophilus influenza type b, chlamydia, varicella-zoster virus or rabies.

Preferred viral immunogenic determinants are such obtained or isolated from e.g. Rotavirus, Foot and mouth disease, Influenza, Parainfluenza, Herpes species, Herpes simplex, Epstein Ban virus, Chicken pox, pseudorabies, Cytomegalovirus, Rabies, Polio, Hepatitis A, Hepatitis B, Hepatitis C, Hepatitis E, Measles, Distemper, Venezuelan equine encephalomyelitis, Feline leukemia virus, Reovirus, Respiratory sycytial virus, Lassa fever virus, Polyoma tumor virus, Canine parvovirus, Papilloma virus, Tick borne encephalitis, Rinderpest, Human rhinovirus species, Enterovirus species, Mengo virus, Paramyxovirus, Avian infectious bronchitis virus, HIV-1, HIV-2, Influenza A and B, LCMV (lymphocytic choriomeningitis Virus), Parovirus, Adenovirus, Togavirus (rubella, yellow fever, dengue fever), Bovine respiratory syncicial virus, and Corona virus, Poxvirus, Herpesvirus, Adenovirus, Papovavirus, Parvovirus, Picornavirus, Togavirus, Myxovirus, Paramyxovirus, Reovirus, RhabdoVirus, Retrovirus, particularly Human Immunodeficient Virus (HIV) and Arenavirus.

Preferred microbial immunogenic determinants are e.g. such obtained or isolated from Achromobacter xylosoxidans, Acinetobacter calcoaceticus, preferably A. anitratus, A. haemolyticus, A. alcaligenes, and A. Iwoffii, Actinomyces israelii, Aeromonas hydrophilia, Alcaligenes species, preferably A. faecalis, A. odorans and A. denitrificans, Arizona hinshawii, Bacillus anthracis, Bacillus cereus, Bacteroides fragilis, Bacteroides melaninogenicus, Bordetella pertussis, Borrelia recurrentis, Brucella species, preferably B. abortus, B. suis, B. melitensis and B. canis, Calymmatobacterium granulomatis, Campylobacter fetus ssp. intestinalis, Campylobacter fetus ssp. jejuni, Chlamydia species, preferably C. psittaci and C. trachomatis, Chromobacterium violaceum, Citrobacter species, preferably C. freundii and C. diversus, Clostridium botulinum, Clostridium perfringens, Clostridium difficile, Clostridium tetani, Corynebacterium diphtheriae, Corynebacterium, preferably C. ulcerans, C. haemolyticum and C. pseudotuberculosis, Coxiella burnetii, Edwardsiella tarda, Eikenella corrodens, Enterobacter, preferably E. cloacae, E. aerogenes, E. hafniae (also named Hafnia alvel) and E. agglomerans, Erysipelothrix rhusiopathiae, Escherichia coli, Flavobacterium meningosepticum, Francisella tularensis, Fusobacterium nucleatum, Gardnerella vaginalis, Haemophilus ducreyi, Haemophilus influenzae, Helicobacter species, Klebsiella species, preferably K. pneumoniae, K. ozaenae og K. rhinoscleromatis, Legionella species, Leptospira interrogans, Listeria monocytogenes, Moraxella species, preferably M. lacunata and M. osloensis, Mycobacterioum bovis, Mycobacterium leprae, Mycobacterium tuberculosis, Mycoplasma species, preferably M. pneumoniae, Neisseria gonorrhoeae, Neisseria meningitidis, Nocardia species, preferably N. asteroides and N. brasiliensis, Pasteurella multocida, Peptococcus magnus, Plesiomonas shigelloides, Proteus species, preferably P. mirabilis, P. vulgaris, P. rettgeri and P. morganii (also named Providencia rettgeri and Morganella morganii respectively), Providencia species, preferably P. alcalifaciens, P. stuartii and P. rettgeri (also named Proteus rettgeri), Pseudomonas aeruginosa, Pseudomonas mallei, Pseudomonas pseudomallei, Rickettsia, Salmonella species, preferably S. enteridis, S. typhi and S. derby, and most preferably Salmonella species of the type Salmonella typhi DT104, Serratia species, preferably S. marcescens, Shigella dysenteriae, S. flexneri, S. boydii and S. sonnei, Spirillum minor, Staphylococcus aureus, Staphylococcus epidermidis, Staphylococcus saprophyticus, Streptobacillus moniliformis, Streptococcus, preferably S. faecalis, S. faecium and S. durans, Streptococcus agalactiae, Streptococcus pneumoniae, Streptococcus pyogenes, Treponema carateum, Treponeam pallidum, Treponema pertenue, preferably T. pallidum, Ureaplasma urealyticum, Vibrio cholerae, Vibrio parahaemolyticus, Yersinia enterocolitica, and Yersinia pestis, Bordetella pertussis, Brucella abortis, Escherichia coli, Salmonella species, salmonella typhi, Streptococci, Vibrio (V. cholera, v. parahaemolyticus), Shigella, Pseudomonas, Brucella species, Mycobacteria species (tuberculosis, avium, BCG, leprosy), Pneumococci, Staphlylococci, Enterobacter species, Rochalimaia henselae, Pasterurella (P. haemolytica, P. multocida), Chlamydia (C. trachomatis, C. psittaci, Lymphogranuloma venereum), Syphilis (Treponema pallidum), Haemophilus species, Mycoplasmosis, Lyme disease (Borrelia burgdorferi), Legionnaires' disease, Botulism (Colstridium botulinum), Corynebacterium diphtheriae, Yersinia entercolitica.

Ricketsial Infectious immunogenic determinants are e.g. such as can be obtained or isolated from Rocky mountain spotted fever, Thyphus, and Ehrlichia.

Parasite and Protozoa immunogenic determinants are e.g. such as can be isolated or obtained from Malaria (Plasmodium falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis), Coccidiosis (Eimeria species).

Fungal immunogenic determinants are e.g immunogenic determinants obtained or isolated from Cryptococcus neoformans, Candida albicans, Apergillus fumigatus, Coccidioidomycosis,

Subunit recombinant protein immunogenic determinants are e.g. immunogenic determinants obtained or isolated from Herpes simplex, Epstein Barr virus, Hepatitis B, Pseudorabies, Flavivirus, Denge, Yellow fever, Neisseria gonorrhoeae, Malaria (circumsporozoite protein, merozoite protein), Trypanosome surface antigen protein, Pertussis, Alphaviruses, and Adenovirus.

Protein immunogenic determinants are e.g. immunogenic determinants obtained or isolated from Diphtheria toxoid, Tetanus toxoid, Meningococcal outer membrane protein (OMP), Streptococcal M protein, Hepatitis B. Influenza hemagglutinin, Cancer antigen, tumor antigens, Toxins, Exotoxins, Neurotoxins, Cytokines and Cytokine receptors, Monokines and monokine receptors.

Cancer antigens may for example be antigens related uveal melanoma, malignant glioma, prostate cancer, skin cancer, liver cancer, breast cancer or colorectal cancer.

Synthetic peptide immunogenic determinants are e.g. immunogenic determinants obtained or isolated from Malaria, Influenza, Foot and mouth disease virus, Hepatitis B, Hepatitis C.

Polysaccharide immunogenic determinants are e.g. immunogenic determinants obtained or isolated from Pneumococcal polysaccharides, Haemophilis influenza, polyribosyl-ribitolphosphate (PRP), Neisseria meningitides, Pseudomonas aeruginosa, Klebsiella pneumoniae.

Oligosaccharide immunogenic determinants are e.g. immunogenic determinants obtained or isolated from Pneumococcal oligosaccharides.

Allergen immunogenic determinants are e.g. immunogenic determinants obtained or isolated from plant pollens, animal dander, dust mites.

In one embodiment of the present invention the immunogenic determinant is derived from a Parvovirus. Said immunogenic determinant could be naturally occuring or it could be synthesized in vitro. For example said immunogenic determinant could be a polypeptide, such as a polypetide comprising the amino acid sequence CDGAVQPDGGQPAVRNER or a derivative thereof. Another example of a preferred immunogenic determinant is recombinant A. salmonicida outer membrane protein (rAsOMP). rAsOMP could for example be produced in E. coli. Preferably, rAsOMP is used at a final purity of ˜75%.

The following examples illustrate the use of a non-conjugated, immunostimulating TGF-^{β} fragment according to the invention for achieving an enhanced immune response. The term “fragment” shall be understood to refer to any immunostimulating TGF-^{β} fragment according to the invention.

The purified fragment may be kept under conditions favouring either monomeric or polymeric molecular variants.

1. A normal final vaccine composition is simply used as solvent for a desired amount (5-1000 microgram dependent on species) of a freeze dried fragment and now used as final vaccine in normal dose or even lower dose.

2. The fragment is dissolved in buffered saline and all other vaccine components successively added until the other vaccine components has the normal desired concentration in the final vaccine used in normal dose or even lower dose.

3. The fragment is added at a vaccine production steps and the rest of other vaccine components are then added until the other vaccine components has the normal desired concentration in the final vaccine used in normal dose or even lower dose.

The immunogenic compositions illustrated herein below may simply be a peptide or protein dissolved in saline, and the immune response resulting after immunisation or vaccination, optionally with and without TGF, for control purposes, may be monitored by any known technique including any suitable form of immunoblotting, such as e.g. any technique involving ELISA or single radial immunediffusion measuring specific antibody titre for the peptide or protein before and after immunisation.

The immune response resulting after immunisation or vaccination—e.g. with and without TGF, for control purposes—may be monitored by e.g. ELISA or single radial immunediffusion measuring specific antibody titre for the components of the complex vaccine, any form of immunoblotting, cytotoxic T cell assay, or survival after challenge with pathogen.

An ELISA analysis preferably comprise analysing serum samples from the immunised/control individual on an ELISA plate according to standard laboratory operations, that comprise coating the plate with the immunogenic determinant, incubation with serial dilutions of serum samples and detection using an antibody specific to Ig's of said individual coupled to an agent that is either directly or indirectly detectable. Such agent could be a fluorescent label, a coloured dye, a radioactive isotope, a heavy metal or an enzymes. Examples of enzymes to be used with ELISA are peroxidases such as horseradish peroxidase, alkaline phosphatase, glucose oxidases, galactosidases and ureases.

The Vaccine Chip technology according to the present invention is further illustrated below. The following examples illustrates how the TGF-^{β} fragment of the present invention may be used in immunogenic compositions including vaccines in preclinical studies using rabbits.

A fragment of TGF-^{β} according to the invention is dissolved in buffered saline and cross-linked moderately by addition of cross-linking glutaraldehyde (gluteraldehyde is a divalent cross-linking compound which covalently attaches the peptides to each other and further fixes the preparation). These methods of conjugating a functional group to a peptide or a protein are well-known to one of ordinary skill in the art. Other chemical cross-linking reagents, chemical coupling reagent, such as charbodiimid, or SH reacting coupling reagents are effective. Also use of biotin-avidin interacting (see e.g. U.S. Pat. No. 5,194,254) may be used.

After a few hours the preperation is dialysed against sterile buffered saline and the cross-linked TGF-^{β} fragment may now be used as a normal carrier protein, and a peptide of choice against which a immune response is desired may be linked to the cross linked TGF-^{β} fragment by other reagents (SH reacting coupling reagents etc.) and dialysed again.

The rabbits are then hyper immunised (see e.g. Ingill and Harboe (1973): Scandinavian Journal of Immunology p 161-164) with the preperation, preferably here with addition of incomplete freunds adjuvant and the antibody-titer of collected antisera measured by suitable well known methods. For the titer against carrier proteins simple radial immundiffusion in agarose gel is suitable, and for the titer against peptide ELISA is suitable. The immunogenic compositions may be tested in other animals such as e.g. mice and swine.

By varying the immunogenic composition for the rabbits cross-linked TGF-^{β} fragment may be substituted by non-cross-linked TGF-^{β}, or with a standard carrier protein such as e.g. ovalbumine, and also the immunogenic determinant against which an immune response is desired, may be conjugated or non-conjugated to the carrier (e.g. ovalbumine or TGF-^{β} preperation. The preferred method for the determinant of choice can be selected based n the resulting antibody titer.

Immunogenic compositions used for contraception (immunological castration) in swine are known in the art and are improved with the vaccine chip technology according to the present invention. Standard methods known to those skilled in the art may be used in preparing the compositions according to the present invention for administration to swine. For example in the simplest formulation, the fragment of TGF-^{β} and a synthetic variant of LHRL of choice (se A. Ladd et al 1990, American Journal of reproductive Immunology 11:56-63), may be dissolved together in sterile saline solution. For long term storage, the polypeptides may be lyophilized and then reconstituted with sterile saline solution or water shortly before administration. Prior to lyophilization, preservatives and other standard additives such as those to provide bulk, e.g., glycine or sodium chloride, may be added. A compatible adjuvant may also be administered with the composition.

The immunological composition according to the present invention is preferably dissolved in sterile saline solution and administered by injection at a dose of several mg of peptide per swine. The composition is preferably administered at 1 to 2 weeks of age and is preferably followed by a booster at 4 to 6 weeks of age. 3 Months after administration the size (weight) of testicles is smaller in the immunised group of swine.

As another example of the many applicabilities of the vaccine chip technology there is provided an improved vaccine against mycoplasma hyopneumoniae infections. Standard methods known to those skilled in the art may be used in preparing the vaccine of the present invention for administration to swine. For example, a fragment of TGF-^{β} according to the invention and the immunogenic determinant of choice from the surface antigens of swine mycoplasma, e.g. as desribed in U.S. 4,894,332, may be dissolved in, sterile saline solution. For long term storage, the polypeptide may be lyophilized and then reconstituted with sterile saline solution or water shortly before administration. Prior to lyophilization, preservatives and other standard additives such as those to provide bulk, e.g. glycine or sodium chloride, may be added. A compatible adjuvant may also be administered with the vaccine.

The vaccine of the present invention is preferably dissolved in sterile saline solution and administered by injection at a dose of several mg of polypeptide from the surface antigens per swine. The vaccine is preferably administered at 1 to 2 weeks of age and is preferably followed by a revaccination or booster at 4 to 6 weeks of age. Vaccinated swine shows lesser clinical signs of mycoplasma infections than unvaccinated.

### EXAMPLES

The following examples should be considered as preferred embodiments of the present invention, however the present invention should not be considered as limited thereto.

### Example 1

The following example demonstrates how the polypeptide having the amino acid sequence:

| | |
|---|---|
| Ala-Leu-Asp-Ala-Ala-Tyr-Cys-Phe-Arg-Asn-Val-Gln-Asp-Asn | (SEQ ID NO:1) |
| -Cys-Cys-Leu-Arg-Pro-Leu-Tyr-Ile-Asp-Phe-Lys-Arg-Asp- | |
| Leu-Gly | |

(hereinafter termed TGF-29) is capable of exerting an immunostimulating effect in an individual.

In particular, the present example demonstrates how the above fragment TGF-29 is capable of converting an otherwise low-immunogenic antigen into a high-immunogenic antigen. The antigen used for the present example is an other-wise low-immunogenic modified polypeptide fragment consisting of about 17 amino acids, originally isolated from a Parvo virus.

Methods and materials

Two different immunisation preparations were made:

i) “Parv”

ii) “Parv+TGF-29”

Both preparations were subcutaneously injected into 2 groups of rabbits. The content of each injected dose of 0.2 mL were:

Parv:

50 microgram parvovirus peptide was dissolved in 0.05 mL PBS and subsequently mixed with 0,05 mL PBS by stirring. The combined fractions were then mixed with 0.1 mL freunds incomplete adjuvant ay stirring.

Parv+TGF-29:

50 microgram parvovirus peptide was dissolved in 0,05 mL PBS and subsequently mixed with 100 micro gram of a crude fraction comprising TGF-29 dissolved in 0,05 mL PBS, and then the combined fractions were mixed with 0,1 mL freunds incomplete adjuvant by stirring.

(Abbreviations: PBS=Phosphate Buffered Saline; crude fraction composition comprising TGF-29 at a purity of about 50%; Parv: Synthesised 18-mer parvo virus peptide derivate acetyl-CDGAVQPDGGQPAVRNER-amide, purity more or about 95% (R831, ID-LELYSTAD, The Netherlands. ref: Langeveld J. P. M., Casal J. I., Osterhaus A. D. M. E., Corter E., de Swart R., Vela C., Dalsgaard K., Puijk W. C., Schaaper W. M. M. and Meloen R. H. (1994). First peptide vaccine providing protection against viral infection in the target animal: Studies of canine parvovirus in dogs. J. Virology 68: 4506-4513.)).

The rabbits were immunised by subcuteous injection according to normal standard immunisation procedures for antibody production in rabbits at a legal laboratory animal facility.

Before immunisation, a pre-immunisation serum sample (T₀) of each rabbit were collected. Two weeks after the last of 3 immunisations (given at 2 week intervals) an immune serum sample (T₁) was collected from each rabbit. Hereafter further immune serum samples were collected T₂ (4 weeks after T₁); T₃ (four weeks after T₂) etc. None of the rabbits showed any unusual clinical signs.

The serum samples were analysed on an ELISA plate according to standard laboratory operations: Coating the plate bottom with parvo virus peptide derivative overnight, washings, incubation with serial dilutions of all serum samples, washings, incubation with HRP-Goat anti Rabbit Ig, washings, incubation with substrate, stop reaction with acid, and reading of the plate.

Results

For all T₀ Sera:

Non-detectable or very low antibody titre to the parvovirus peptide.

For Immune serum-samples of the “Parv” Group

Non-detectable or less antibody titre to the parvovirus peptide than in the corresponding T₀ sera.

For Immune Sera Samples of the “Parv+TGF-29” Group:

Rabbit 146 shows higher antibody titre to the parvovirus peptide (up to 5 to 7 two-fold dilutions more than the corresponding T₀ serum)

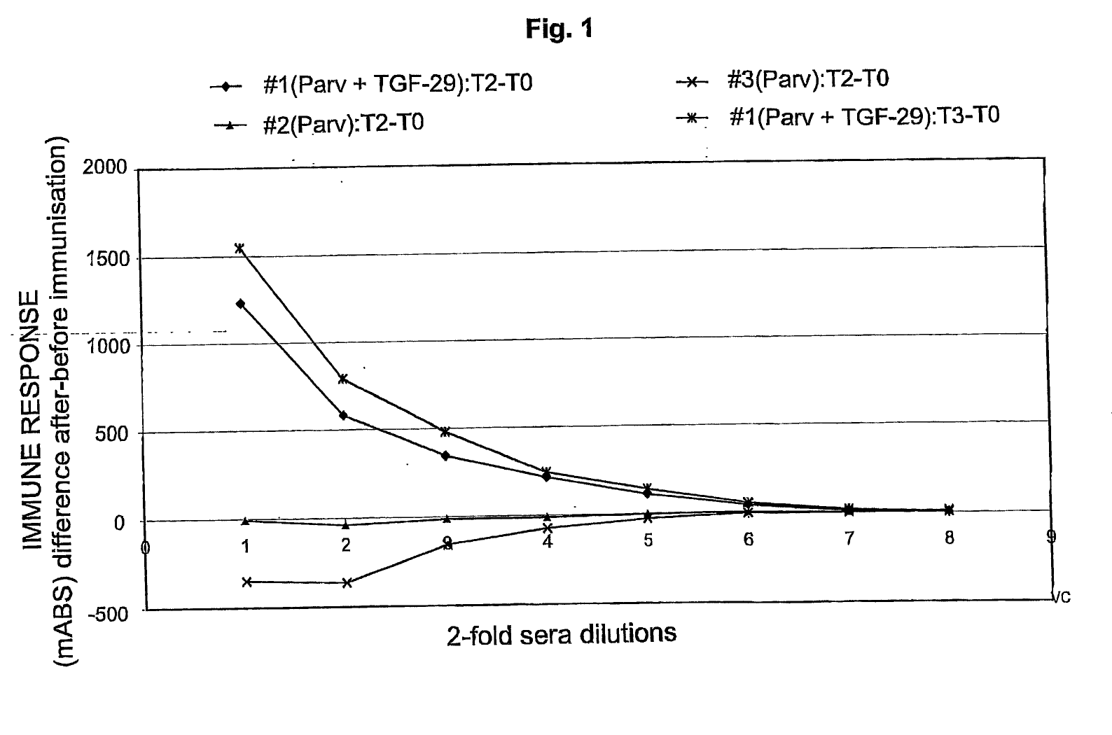

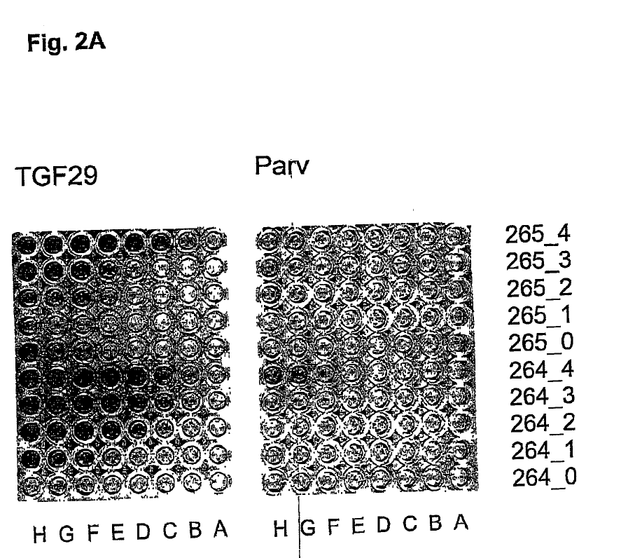

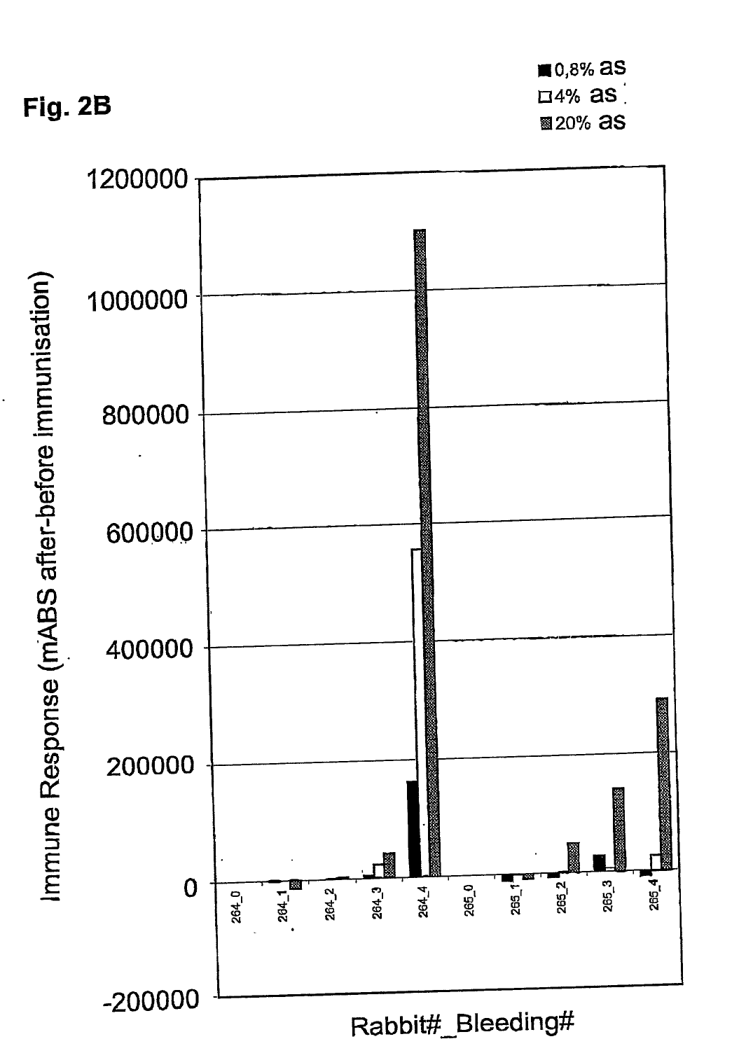

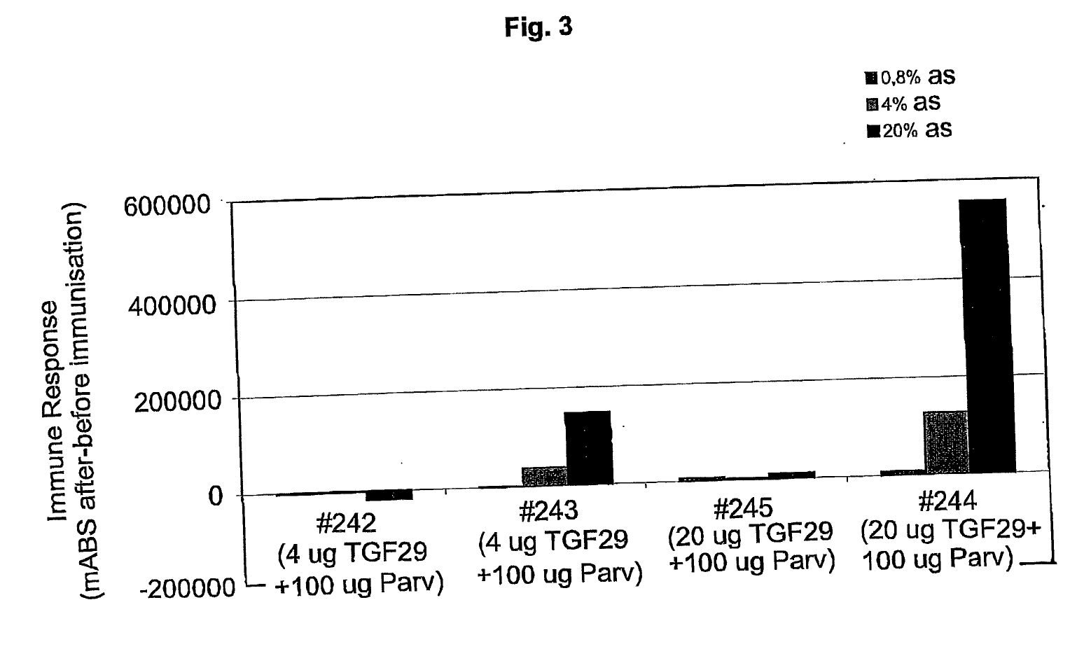

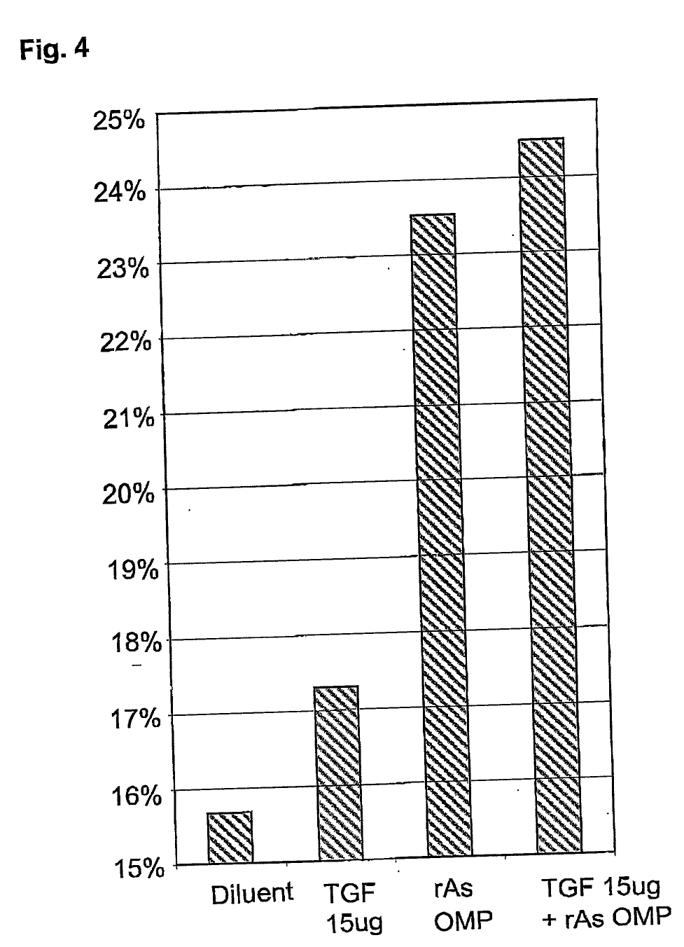

The results were quantified by using an ELISA reader and measuring the resulting absorbance. The results are disclosed in FIG. 1.

Conclusions

This example demonstrates that the TGF-29 fragment illustrated herein above—even in a very simple application involving mixing a solution of TGF-29 with a low-immunogenic solution of a model antigen by stirring—is capable of enhancing the immune response to the otherwise low-immunogenic model antigen.

### Example 2

The following example demonstrates also how TGF29 is capable of exerting an immunostimulating effect.

In particular, the example demonstrates how the above fragment TGF-29 is capable of converting an otherwise low-immunogenic antigen into a high-immunogenic antigen after chemical conjugation to the low-immunogenic antigen. The antigen used for the present example is the same modified polypeptide fragment as in example 1, originally isolated from a Parvo virus.

Methods and Materials

One immunisation preparation were made: “TGF-29-Parv conjugate”

The preparation was subcutaneously injected into 2 groups of rabbits,

The content of each injected dose of 0,2 mL were prepared in 2 ml batch volumina as follows:

A small 10 ml flat-bottomed clean glass container with a diameter about 2 cm and a small (1 cm long) magnetic Teflon coated clean rod inside was firmly secured above a rotating (600-800 rpm) magnetic stirring equipment at room temperature

First 0,8 mL Tris-HCl (50 mM, pH about 8.5) was added.

Second 0,1 mL was added of a freshly thawed TGF-29 stock solution comprising of 5 milligram purified TGF-29 dissolved in 1 mL H₂0 and normally stored below −18° Celcius.

Third, after about 2 hours stirring in atmospheric air at room temperature, 0, 1 mL was added of a freshly thawed parvovirus peptide stock solution comprising 5 milligram parvovirus peptide dissolved in 1 mL H₂O and normally stored below −18° Celcius.

The stirring was continued over night in atmospheric air at room temperature.

Next day the otherwise clear solution was now slightly opal whitish due to the oxidative induced cross-links of the 1 mL peptide conjugate solution.

Finally the peptide conjugate solution (1 mL ) was mixed with 1 mL freunds incomplete adjuvant by stirring. The resulting batch of “TGF-29—Parv conjugate” was stored below −18° Celcius when not in use for injection. Each injected dose contains stochiometrically the same amount of Freunds incomplete adjuvant and total amount of TGF-29 or total amount of parvovirus peptide as the immunisation preparation “Parv+TGF-29” of example 1.

(Abbreviations: Tris-HCl: Tris is a common trade name for a commercially (eg.Sigma) available buffer salt solution adjusted with H₂O and HCl to desired pH and molarity; purified TGF-29=composition comprising TGF-29 at a purity of about 95%; Parv: Synthesised 1 8-mer parvo virus peptide derivate acetyl-CDGAVQPDGGQPAVRNER-amide, purity more or about 95% (R831, IDLELYSTAD, The Netherlands, ref: Langeveld J. P. M., Casal J. I., Osterhaus A. D. M. E., Corter E., de Swart R.,Vela C., Dalsgaard K., Puijk W. C., Schaaper W. M. M. and Meloen R. H. (1994). First peptide vaccine providing protection against viral infection in the target animal: Studies of canine parvovirus in dogs, J. Virology 68:4506-4513)).

The rabbits were then immunised by subcutaneous injection according to the normal standard immunisation procedure for antibody production in rabbits at a legal laboratory animal facility.

Before immunisation, a pre-immunisation serum sample (T₀) of each rabbit were collected. Before each of the following immunisations (given at 2 week intervals) an immune serum sample (T₁, T₂, T₃ or T₄ ) was collected from each rabbit. The serum sampling frequency is higher compared with example 1. None of the rabbits showed any unusual clinical signs.

The serum samples were analysed on an ELISA plate according to standard laboratory operations: Coating the plate bottom with parvo virus peptide derivative overnight, washings, incubation with serial dilutions of all serum samples, washings, incubation with HRP-Goat anti Rabbit Ig, washings, incubation with substrate, stop reaction with acid, and reading of the plate.

Results

For all T₀ Sera:

Non-detectable or very low antibody titre to the parvovirus peptide.

For Immune Sera Samples:

Four to eight weeks after first immunisation all immune serum samples shows higher antibody titre to the parvovirus peptide than the corresponding T₀ serum (up to 3 five-fold dilutions more than the corresponding T₀ serum). This is in clear contrast to the control group “parv” of former experiment.

Furthermore, the immune sera have a significant increase in antibody titers to the TGF29 fragment (up to 7 five-fold dilutions more than the corresponding T₀ serum).

The results were quantified by using an ELISA reader and measuring the resulting absorbance. The results are disclosed in table 1 and in FIG. 2A and FIG. 2B. Table 1 discloses the absorbance as measured by the ELISA reader, when testing the antibody titer of bleeding 0-4 of rabbit #264 and rabbit #265, using either the parvovirus peptide “parv” or TGF29 as target and FIG. 2A shows a picture of the corresponding ELISA plate. FIG. 2B is a graphic illustration of the results

Conclusions

The simple experiments show that the TGF-29 fragment illustrated herein above—even in a very simple conjugation application involving overnight mild oxidative cross linking of TGF-29 with a low-immunogenic solution of a model antigen by stirring in atmospheric air—is capable of enhancing the immune response to the otherwise low-immunogenic model antigen.

TABLE 1
TGF29-Parv conjugate. ELISA Reading 15/2-2001, 490 nanometer, UNITS: ABS × 1000000

| Elisa | | | | | | | | | 0,8% as | 4% as | 20% as | |
|---|---|---|---|---|---|---|---|---|---|---|---|---|
| plate # | Row | Bleeding | Rabbit_bleeding | Target | A | B | C | D | E | F | G | H |
| 3 | 1 | 0 | 264_0 | TGF29 | 36000 | 36000 | 37000 | 40000 | 54000 | 180000 | 437000 | 255000 |
| 3 | 2 | 1 | 264_1 | TGF29 | 36000 | 36000 | 41000 | 114000 | 480000 | 1399000 | 2433000 | 3472000 |
| 3 | 3 | 2 | 264_2 | TGF29 | 40000 | 78000 | 380000 | 1266000 | 2993000 | (+) | (+) | (+) |
| 3 | 4 | 3 | 264_3 | TGF29 | 107000 | 385000 | 1459000 | 3135000 | (+) | (+) | (+) | (+) |
| 3 | 5 | 4 | 264_4 | TGF29 | 213000 | 748000 | 2325000 | (+) | (+) | (+) | (+) | 3178000 |
| 3 | 6 | 0 | 265_0 | TGF29 | 38000 | 36000 | 37000 | 67000 | 298000 | 875000 | 1337000 | 2706000 |
| 3 | 7 | 1 | 265_1 | TGF29 | 43000 | 33000 | 33000 | 34000 | 99000 | 285000 | 317000 | 628000 |
| 3 | 8 | 2 | 265_2 | TGF29 | 42000 | 38000 | 39000 | 153000 | 537000 | 1267000 | 1294000 | 1422000 |
| 3 | 9 | 3 | 265_3 | TGF29 | 40000 | 35000 | 55000 | 259000 | 1029000 | 2468000 | 3332000 | (+) |
| 3 | 10 | 4 | 265_4 | TGF29 | 280000 | 940000 | 2718000 | (+) | (+) | (+) | (+) | 2696000 |
| 6 | 1 | 0 | 264_0 | Parv | 38000 | 37000 | 38000 | 44000 | 37000 | 40000 | 59000 | 225000 |
| 6 | 2 | 1 | 264_1 | Parv | 38000 | 37000 | 37000 | 38000 | 36000 | 40000 | 45000 | 182000 |
| 6 | 3 | 2 | 264_2 | Parv | 38000 | 36000 | 37000 | 38000 | 37000 | 41000 | 61000 | 45000 |
| 6 | 4 | 3 | 264_3 | Parv | 37000 | 37000 | 37000 | 38000 | 41000 | 63000 | 100000 | 451000 |
| 6 | 5 | 4 | 264_4 | Parv | 45000 | 37000 | 41000 | 52000 | 198000 | 596000 | 1161000 | 1344000 |
| 6 | 6 | 0 | 265_0 | Parv | 41000 | 43000 | 38000 | 47000 | 49000 | 41000 | 59000 | 178000 |
| 6 | 7 | 1 | 265_1 | Parv | 41000 | 40000 | 40000 | 40000 | 38000 | 40000 | 51000 | 82000 |
| 6 | 8 | 2 | 265_2 | Parv | 45000 | 44000 | 40000 | 43000 | 43000 | 42000 | 109000 | 311000 |
| 6 | 9 | 3 | 265_3 | Parv | 43000 | 45000 | 40000 | 43000 | 76000 | 47000 | 198000 | 562000 |
| 6 | 10 | 4 | 265_4 | Parv | 48000 | 43000 | 49000 | 41000 | 40000 | 67000 | 347000 | 438000 |

(+): Overflow

### Example 3

The following example demonstrates also how TGF-29 is capable of exerting an immunostimulating effect.

In particular, the example demonstrates how increasing doses of the above fragment TGF-29 is capable of converting an otherwise low-immunogenic antigen into a high-immunogenic antigen after simple mixing with the low-immunogenic antigen and Titermax adjuvant. The antigen used for the present example is the same modified polypeptide fragment as in example 1, originally isolated from a Parvo virus.

Methods and Materials

Two different immunisation preparations were made:

iii) “Viol″”

iv) “Iris”

Both preparations were subcutaneously injected into 2 groups of rabbits. Each injected dose was 0, 1 mL and the preparations for a resulting volume between 0,4 and 0,5 mL was made as follows:

Viol:

Into a small 1, 8 mL plast vial 80 microliter was added of a freshly thawed parvovirus peptide stock solution comprising 5 milligram parvovirus peptide dissolved in 1 mL H₂O and normally stored below −18° Celcius.

Into the same vial 3,2 microliter was added of a freshly thawed TGF29 stock solution comprising of 5 milligram purified TGF-29 dissolved in 1 mL H₂O and normally stored below −18 Celcius.

Into the same vial 166,8 microliter H₂O was added and the combined fractions subsequently mixed by stirring a few seconds and then transferred in drops, while stirring, into a small 1,8 mL plast vial preloaded with 250 microliter Titermax. The resulting volume of the water-in-oil emulsion volume was a little more than 0,4 mL.

Each injected dose of 0,1 mL therefore contains approximate 4 ug TGF-29 and 100 ug parvovirus peptide.

Iris:

Into a small 1, 8 mL plast vial 80 microliter was added of a freshly thawed parvovirus peptide stock solution comprising of 5 milligram parvovirus peptide dissolved in 1 mL H₂O and normally stored below −18° Celcius.

Into the same vial 16 microliter was added of a fresh thawed TGF-29 stock solution comprising of 5 milligram purified TGF-29 dissolved in 1 mL H₂O and normally stored below −18° Celcius.

Into the same vial 154 microliter H₂O was added and the combined fractions subsequently mixed by stirring a few seconds and then transferred in drops, while stirring, into a small 1,8 mL plast vial preloaded with 250 microliter Titermax adjuvant. The resulting volume of the water-in-oil emulsion volume was a little more than 0,4 mL. Each injected dose of 0,1 mL therefore contains approximate 20 ^{μ}g TGF-29 and 100 ^{μ}g parvovirus peptide.

(Abbreviations: Titermax adjuvant: Titermax Classical Adjuvant, Product no H4397, SIGMA-ALDRICH DENMARK A/S; Purified TGF29=composition comprising TGF-29 at a purity of about 95%; Parv: Synthesised 18-mer parvo virus peptide derivate acetyl-CDGAVQPDGGQPAVRNER-amide, purity more or about 95% (R831, IDLELYSTAD, The Netherlands, ref: Langeveld J. P. M., Casal J. I., Osterhaus A. D. M. E., Corter E., de Swart R.,Vela C., Dalsgaard K, Puijk W. C., Schaaper W. M. M. and Meloen R. H. (1994). First peptide vaccine providing protection against viral infection in the target animal: Studies of canine parvovirus in dogs. J. Virology 68:4506-4513.)).

The rabbits were then immunised according to the normal standard immunisation procedure for antibody production in rabbits at a legal laboratory animal facility.

Before immunisation, a pre-immunisation serum sample (T₀) of each rabbit was collected. Before each of the following immunisations (given at 2 week intervals) an immune serum sample (T₁, T₂, or T₃) was collected from each rabbit. None of the rabbits showed any unusual clinical signs.

The serum samples were analysed on an ELISA plate according to standard laboratory operations: Coating the plate bottom with parvo virus peptide derivative overnight, washings, incubation with serial dilutions of all serum samples, washings, incubation with HRP-Goat anti Rabbit Ig, washings, incubation with substrate, stop reaction with acid, and reading of the plate.

Results

Of all bleedings in this example, the highest titer to the parvovirus peptide were bleeding T₃ (6 weeks after first immunisation) of a rabbit belonging to the group injected with the highest dose TGF-29 (20 ug). This bleeding were more than three 5-fold serial antiserum dilutions better than the corresponding T₀ serum of the rabbit.

The results were quantified by using an ELISA reader and measuring the resulting absorbance. The results are disclosed in FIG. 3.

Conclusions

The simple dose experiment show that the TGF-29 fragment illustrated herein above—also when combined with other adjuvants than freunds incomplete adjuvant—is capable of enhancing the immune response to the otherwise low-immunogenic model antigen.

### Example 4

The following example demonstrates how TGF-29 is capable of exerting an immunostimulating effect when used in a fish vaccine.

In particular, the example demonstrates how vaccination involving the above fragment TGF-29 is capable of increasing the survival of fish when these, after vaccination, are experimentally challenged with the causative agent for salmonid furunculosis (Aeromonas salmonicida) at a legal experimental animal facility.

Methods and Materials

References to detailed examples of how experimental challenge of fish can be done are found in: Kaastrup, P., Hørlyck, V., Olesen, N. J., Lorenzen, N., Vestergaard-Jørgensen, P. E. and Berg, P. :Paternal association of increased susceptibility to viral haemorrhacic septicaemia (VHS) in rainbow trout (Oncorhynchus mykiss). (Canadian Journal of Fisheries and Aquatic Science, 48, 1188-1192, 1991)

An experimental recombinant protein vaccine for furunculosis was used as a model to test the adjuvant effect of TGF-29. Four different placebo or test vaccines (A, B, C and D) were tested for efficacy 350 degree days (mean water temperature×#days) after intraperitonal injection of 60 fish (Atlantic Salmon) per group were made. The composition of each vaccine group was:

A. Diluent/oil-in-water adjuvant (negative control)

B. 15 ug purified TGF-29+Diluent/oil-in-water adjuvant

C. rAsOMP+Diluent/oil-in-water adjuvant

D. 15 ug purified TGF-29+rAsOMP+Diluent/oil-in-water adjuvant

350 degree days post-vaccination, blood samples were collected from ten fish per group for later measurement of antibody titres to the TGF-29 and to the model antigen rAsOMP of the virulent pathogen by ELISA. Also at this time, 50 fish per group were subjected to lethal challenge with a standard dose of virulent A. salmonicida. The survival of fish in all groups was monitored for 17 days post-challenge, with mortalities collected daily. The cause of mortality was examined in every fish by standard microbiological culture methods.

(Abbreviations: Purified TGF-29=composition comprising TGF-29 at a purity of about 95%; rAsOMP=recombinant A. salmonicida outer membrane protein produced in E. coli at a final purity of ˜75%)

Results

The results of the A. salmonicida challenge trial are disclosed in FIG. 4 showing the resulting survival at day 17. The survival is highest for fish vaccinated with both TGF-29 and the model antigen rAsOMP

Conclusions

This example shows that the TGF-29 fragment, illustrated herein above,—when combined with adjuvants other than freunds incomplete adjuvant or Titermax, and with an other model antigen (rAsOMP)—is capable of enhancing the immune response to an antigen. Also, the data demonstrates an enhanced immune response in fish resulting in survival after challenge. It is important to notice that TGF-29 exerts its effect in animals as phylogenetically diverse as mammals and fish. It is therefore concluded, as the sequence also is very conservative, that TGF-29 exerts the described effects in all vertebrates from fish to Man.

1. An immunogenic composition comprising
i) at least one fragment of TGF-^{β} capable of eliciting an immunostimulating effect in an individual, and
ii) at least one immunogenic determinant against which it is desirable to elicit an immunogenic response,
wherein said at least one fragment of TGF-^{β} and said at least one immunogenic determinant are not identical.

2. The immunogenic composition according to claim 1, wherein said fragment of TGF-^{β} comprises the amino acid sequence

| | |
|---|---|
| Ala-Leu-Asp-Ala-Ala-Tyr-Cys-Phe-Arg-Asn-Val-Gln-Asp- | (SEQ ID NO: 1) |
| Asn-Cys-Cys-Leu-Arg-Pro-Leu-Tyr-Ile-Asp-Phe-Lys-Arg- | |
| Asp-Leu-Gly | |

including any functional equivalents thereof obtained by addition, substitution or deletion of at least one amino acid.

3. The immunogenic composition according to claim 1, wherein said fragment of TGF-^{β} essentially consists of the amino acid sequence

| | |
|---|---|
| Ala-Leu-Asp-Ala-Ala-Tyr-Cys-Phe-Arg-Asn-Val-Gln-Asp- | (SEQ ID NO: 1) |
| Asn-Cys-Cys-Leu-Arg-Pro-Leu-Tyr-Ile-Asp-Phe-Lys-Arg- | |
| Asp-Leu-Gly | |

including any functional equivalents thereof obtained by addition, substitution or deletion of at least one amino acid.

4. The immunogenic composition according to claim 1, wherein said fragment of TGF-^{β} consists of the amino acid sequence

| | |
|---|---|
| Ala-Leu-Asp-Ala-Ala-Tyr-Cys-Phe-Arg-Asn-Val-Gln-Asp- | (SEQ ID NO: 1) |
| Asn-Cys-Cys-Leu-Arg-Pro-Leu-Tyr-Ile-Asp-Phe-Lys-Arg- | |
| Asp-Leu-Gly | |

including any functional equivalents thereof obtained by addition, substitution or deletion of at least one amino acid.

5. The immunogenic composition according to claim 1, wherein said fragment of TGF-^{β} essentially consists of the amino acid sequence

| | |
|---|---|
| Ala-Leu-Asp-Ala-Ala-Tyr-Cys-Phe-Arg-Asn-Val-Gln-Asp- | (SEQ ID NO: 1) |
| Asn-Cys-Cys-Leu-Arg-Pro-Leu-Tyr-Ile-Asp-Phe-Lys-Arg- | |
| Asp-Leu-Gly | |

6. The immunogenic composition according to any of claims 1 to 4, wherein said substitution is a conservative amino acid substitution.

7. The immunogenic composition according to claim 6, wherein said glycine (Gly) of said fragment of TGF-^{β} is substituted with an amino acid selected from the group of amino acids consisting of Ala, Val, Leu, and Ile.

8. The immunogenic composition according to claim 6, wherein at least one of said alanines (Ala) of said fragment of TGF-^{β} is substituted with an amino acid selected from the group of amigo acids consisting of Gly, Val, Leu, and Ile.

9. The immunogenic composition according to claim 6, wherein said valine (Val) of said fragment of TGF-^{β} is substituted with an amino acid selected from the group of amino acids-consisting of Gly, Ala, Leu, and Ile.

10. The immunogenic composition according to claim 6, wherein at least one of said leucines (Leu) of said fragment of TGF-^{β} is substituted with an amino acid selected from the group of amino acids consisting of Gly, Ala, Val, and Ile.

11. The immunogenic composition according to claim 6, wherein said isoleucine (Ile) of said fragment of TGF-^{β} is substituted with an amino acid selected from the group of amino acids consisting of Gly, Ala, Val and Leu.

12. The immunogenic composition according to claim 6, wherein at least one of said aspartic acids (Asp) of said fragment of TGF-^{β} is substituted with an amino acid selected from the group of amino acids consisting of Glu, Asn, and Gln.

13. The immunogenic composition according to claim 6, wherein at least one of said phenylalanines (Phe) of said fragment of TGF-^{β} is substituted with an amino acid selected from the group of amino acids consisting of Tyr, Trp, His, Pro, and preferably selected from the group of amino acids consisting of Tyr and Trp.

14. The immunogenic composition according to claim 6, wherein at least one of said tyrosines (Tyr) of said fragment of TGF-^{β} is substituted with an amino acid selected from the group of amino acids consisting of Phe, Trp, His, Pro, and preferably selected from the group of amino acids consisting of Phe and Tip.

15. The immunogenic composition according to claim 6, wherein at least one of said arginines (Arg) of said fragment of TGF-^{β} is substituted with an amino acid selected from the group of amino acids consisting of Lys and His.

16. The immunogenic composition according to claim 6, wherein said lysine (Lys) of said fragment of TGF-^{β} is substituted with an amino acid selected from the group of amino acids consisting of Arg and His.

17. The immunogenic composition according to claim 6, wherein at least one of said asparagines (Asn) of said fragment of TGF-^{β} is substituted with an amino acid selected from the group of amino acids consisting of Asp, Glu, and Gln.

18. The immunogenic composition according to claim 6, wherein said glutamine (Gln) of said fragment of TGF-^{β} is substituted with an amino acid selected from the group of amino acids consisting of Asp, Glu, and Asn.

19. The immunogenic composition according to claim 6, wherein said proline (Pro) of said fragment of TGF-^{β} is substituted with an amino acid selected from the group of amino acids consisting of Phe, Tyr, Trp, and His.

20. The immunogenic compassion according to claim 6, wherein at least one of said cysteines (Cys) of said fragment of TGF-^{β} is substituted with an amino acid selected from the group of amino acids consisting of Asp, Glu, Lys, Arg, His, Asn, Gln, Ser, Thr, and Tyr.

21. The immunogenic composition according to any of the preceding claims, wherein said immunostimulating effect is characterised by an enhanced antibody response.

22. The immunogenic composition according to any of the claims 1-20, wherein said immunostimulating effect is characterised by a cytotoxic response.

23. The immunogenic composition according to claim 21, wherein said response is caused by an increase in at least one class of immunoglobulins.

24. The immunogenic composition according to claim 23, wherein said increase involves a plurality of immunoglobulin classes.

25. The immunogenic composition according to claim 23, wherein said response is caused by an increase in the, level of T-cells.

26. The immunogenic composition according to claim 23, wherein said response is caused by an increase in the level of cytotoxic T-cells.

27. The immunogenic composition according to claim 1, wherein said fragment and said immunogenic determinant are both non-conjugated.

28. The immunogenic composition according to claim 1, wherein said fragment is conjugated and said immunogenic determinant is non-conjugated.

29. The immunogenic composition according to claim 1, wherein said fragment is non-conjugated and said immunogenic determinant is conjugated.

30. The immunogenic composition according to claim 1, wherein said fragment and said immunogenic determinant are both conjugated.

31. The immunogenic composition according to claim 30, wherein said fragment is conjugated to said immunogenic determinant.

32. The immunogenic composition according to claim 1 further comprising a carrier.

33. The immunogenic composition according to claim 32, wherein said fragment and said immunogenic determinant are both non-conjugated.

34. The immunogenic composition according to claim 33, wherein said carrier is non-conjugated.

35. The immunogenic composition according to claim 33, wherein said carrier is conjugated.

36. The immunogenic composition according to claim 32, wherein said fragment is conjugated and said immunogenic determinant is non-conjugated.

37. The immunogenic composition according to claim 36, wherein said carrier is non-conjugated.

38. The immunogenic composition according to claim 36, wherein said carrier is conjugated

39. The immunogenic composition according to claim 38, wherein said fragment is conjugated to said carrier.

40. The immunogenic composition according to claim 32, wherein said fragment is non-conjugated and said immunogenic determinant is conjugated.

41. The immunogenic composition according to claim 40, wherein said carrier is non-conjugated.

42. The immunogenic composition according to claim 40, wherein said carrier is conjugated

43. The immunogenic composition according to claim 42, wherein said immunogenic determinant is conjugated to said carrier.

44. The immunogenic composition according to claim 32, wherein said fragment and said immunogenic determinant are both conjugated.

45. The immunogenic composition according to claim 44, wherein said fragment is conjugated to said immunogenic determinant.

46. The immunogenic composition according to any of claims 44 and 45, wherein said carrier is non-conjugated.

47. The immunogenic composition according to any of claims 44 and 45, wherein said carrier is conjugated.

48. The immunogenic composition according to claim 47, wherein said fragment is conjugated to said carrier.

49. The immunogenic composition according to claim 47, wherein said immunogenic determinant is conjugated to said carrier.

50. The immunogenic composition according to claim 47, wherein said carrier is conjugated to said fragment and to said immunogenic determinant.

51. The immunogenic composition according to any of claims 1 to 50 further comprising an adjuvant.

52. The immunogenic composition according to any of claims 1 to 4, wherein said addition or deletion is an addition or a deletion of from 2 to preferably 10 amino acids, such as from 2 to 8 amino acids, for example from 2 to 6 amino acids, such as from 2 to 4 amino acids.

53. A vaccine comprising the immunogenic composition according to any of claims 1 to 52.

54. The immunogenic composition according to any of claims 1 to 52 or vaccine according to claim 53 for use in a method of immunising an individual in need of immunisation.

55. The immunogenic composition according to any of claims 1 to 52 or vaccine according to claim 53 for use in a method of immunising an individual in need of immunisation, said method comprising the steps of
i) providing said immunogenic composition or said vaccine, and
ii) administering said immunogenic composition or said vaccine to said individual.

56. A fragment of TGF-^{β} capable of facilitating an immunostimulating effect in an individual for use as a medicament.

57. A method of immunising, an individual in need of immunisation, said method comprising the steps of
i) providing an immunogenic composition according to any of claims 1 to 52, or a vaccine according to claim 53, and
ii) administering said immunogenic composition or said vaccine to said individual.

58. The method according to claim 57, wherein at least 50% of individuals exposed to said immunogenic composition or vaccine are immunised.

59. Use of a fragment of TGF-^{β} capable of facilitating an immunostimulating effect in an individual in the manufacture of an immunogenic composition or a vaccine.

60. Use according to claim 59 for the manufacture of a medicament for enhancing the immunostimulating effect of an immunisation.
